(19) United States
(12) Patent Application Publication
(10) Pub. No.: US 2026/0137882 A1
(43) Pub. Date: May 21, 2026

(54) ADAPTER FOR PRESSURE VALVE

(71) Applicant: Fisher & Paykel Healthcare Limited, Auckland (NZ)

(72) Inventors: Timothy Richard RUTLEDGE, Auckland (NZ); Evan Anthony MAUNDER, Auckland (NZ); Mitchell Ryan Cammell OXENBRIDGE, Auckland (NZ)

(21) Appl. No.: 18/706,221

(22) PCT Filed: Dec. 13, 2022
(86) PCT No.: PCT/IB2022/062098

§ 371 Date: Apr. 30, 2024

Related U.S. Application Data
(60) Provisional application No. 63/265,317, filed on Dec. 13, 2021.

(51) Int. Cl. A61M 16/00; A61M 16/06; A61M 16/08; A61M 16/10; A61M 16/16; A61M 16/20

(52) U.S. Cl. CPC A61M 16/0003; A61M 16/0672; A61M 16/0875; A61M 16/107; A61M 16/161; A61M 16/206; A61M 16/209; A61M 2016/0027; A61M 2016/003; A61M 2202/0208; A61M 2205/3334; A61M 2205/584; A61M 2205/6081

(57) ABSTRACT

The invention is directed to an adapter comprising a rigid base portion defining an outlet, and a head portion defining an inlet. The head portion is configured for removable attachment to the rigid base portion such that a gas flow passage is provided between the inlet and the outlet when the head portion is attached to the rigid base portion. The adapter is configured to provide a first flow restriction for restricting gas flow through the gas flow passage, and an access passage to the gas flow passage. The access passage is provided downstream of the first flow restriction.

### TECHNICAL FIELD

The present disclosure generally relates to an adapter for a pressure valve in medical systems for conveying gases to and/or from a patient. A pressure valve may include an apparatus that controls pressure in a system, including for example, a pressure regulator or a pressure relief device. In particular, the adapter may be calibrated for paediatric use, although the scope of the invention is not necessarily limited thereto.

### BACKGROUND

Respiratory gas supply systems provide gas for delivery to a patient. Respiratory gas supply systems typically include a fluid connection between a gas supply and the patient. This may include an inspiratory tube and a patient interface. Such systems include a number of different components to ensure gas is correctly delivered to a patient. Many of the components are single use components that are disposed of after each use, while other components are multi-use components. In some situations, multi-use components are preferred. In some situations, it is necessary to connect single use components to multi-use components. However, this can cause problems if single use components are incorrectly or inadvertently assembled with multi-use components. In addition, some components are complex products that have a number of different features and functions. The design and/or production of such components cannot be readily altered or modified.

Embodiments of the invention may provide an adapter which enables connection between single-use and multi-use components in a respiratory gas supply system.

A reference herein to a patent document or any other matter identified as prior art, is not to be taken as an admission that the document or other matter was known or that the information it contains was part of the common general knowledge as at the priority date of any of the claims.

### SUMMARY OF THE INVENTION

According to one aspect of the invention, there is provided an adapter comprising
a rigid base portion defining an outlet, and
a head portion defining an inlet, the head portion being configured for removable attachment to the rigid base portion such that a gas flow passage is provided between the inlet and the outlet when the head portion is attached to the rigid base portion,
wherein the adapter is configured to provide
   a first flow restriction for restricting gas flow through the gas flow passage, and
   an access passage to the gas flow passage, the access passage being provided downstream of the first flow restriction.

The access passage may be provided by an aperture in the head portion. In some embodiments, access passage may be provided by a plurality of apertures in the head portion. The one or more apertures may be provided in a stepped portion of the head portion.

The first flow restriction may be provided at the inlet. The degree of flow restriction may be based on the size of an opening at the inlet.

The head portion may be tethered to the rigid base portion.

The head portion may be tapered. In particular, the head portion may be tapered towards the inlet.

The head portion may be configured for sealing engagement with a connector associated with a pressure valve.

A pressure valve controls pressure in a system such as a respiratory system. In some embodiments, a pressure valve may be a pressure regulator or pressure relief device. A pressure relief device may be a pressure relief valve (PRV) or more specifically a flow compensated pressure relief valve (FCPRV).

In particular, the head portion may be configured to form a cavity with the connector when the head portion is engaged with the connector.

Moreover, the cavity may be in fluid communication with the gas flow passage via the access passage.

The rigid base portion may include one or more projections for engagement with one or more corresponding recesses provided by the head portion, for removable attachment thereto. Alternatively, the rigid base portion may include one or more recesses for receiving one or more corresponding projections provided by the head portion, for removable attachment thereto.

In one embodiment, the head portion may be removably attached to the rigid base portion by any suitable means, for example friction fit or press fit, via complimentary threaded portions or the like, or any combination thereof.

The rigid base portion may be configured for attachment to a flexible conduit at the outlet of the rigid base portion.

The adapter may further comprise
a second head portion configured for removable attachment to the rigid base portion, the second head portion defining an inlet such that a gas flow passage is provided between the inlet of the second head portion and the outlet of the rigid base portion when the second head portion is attached to the rigid base portion,
wherein the adapter is configured to provide a second flow restriction for restricting gas flow through the gas flow passage when the second head portion is attached to the base portion, and
wherein the second flow restriction is different to the first flow restriction.

The adapter may be configured to provide an access passage to the gas flow passage when the second head portion is attached to the rigid base portion, the access passage being provided downstream of the second flow restriction.

Typically, the flow restriction is provided by the inlet of the head portion. In particular, a smaller opening at the inlet provides more flow restriction than a larger opening at the inlet. In one embodiment, the inlet of the second head portion may be smaller than the inlet of the first head portion. In other words, the opening at the inlet of the second head portion may be smaller than the opening at the inlet of the first head portion.

According to another aspect of the invention, there is provided an adapter comprising
a hollow body defining an inlet and an outlet configured to provide a gas flow passage therethrough,
a flow restriction configured to restrict gas flow through the gas flow passage,
an access passage to the gas flow passage, the access passage being provided downstream of the flow restriction, and
an attachment portion proximate the outlet configured for removable attachment to a rigid adapter base portion.

The hollow body may define an aperture downstream of the inlet for providing the access passage to the gas flow passage.

The flow restriction may be provided at the inlet. The hollow body may be tapered towards the inlet.

The adapter may further include an engagement portion configured for sealing engagement with a connector associated with a pressure valve.

The hollow body may be configured to form a cavity with the connector when the engagement portion is engaged with the connector.

The cavity may be in fluid communication with the gas flow passage via the access passage.

The attachment portion may include one or more recesses for engagement with one or more projections of the rigid base portion for removable attachment thereto.

The adapter may further include one or more extensions extending outwardly from the hollow body to prevent attachment of a like adapter over the hollow body of the adapter.

The one or more extensions may include a projection extending radially outwardly from an external surface of the hollow body. Alternatively, or in combination, the one or more extensions may include one or more projections extending outwardly in a lengthwise direction along the hollow body.

According to a further aspect of the invention, there is provided an adapter assembly including
one or more adapters, each adapter being an adapter as described above,
a rigid adapter base portion for removable attachment to the one or more adapters,
wherein the flow restriction of each adapter is different.

The flow restriction may be provided by the size of the opening at the inlet. To provide different flow restrictions, the inlet size of each adapter may be different.

The adapter assembly may further include a tether for tethering one or more of the adapters to the base portion.

Each of the one or more adapters and the rigid base portion may include a channel for receiving the tether.

The rigid base portion may be configured for connection to a flexible conduit.

According to another aspect of the invention, there is provided an adapter comprising
a hollow body defining an inlet and an outlet for providing a gas flow passage therethrough,
a flow restriction for restricting gas flow through the gas flow passage,
an access passage to the gas flow passage, the access passage being provided downstream of the flow restriction, and
a mounting portion proximate the outlet for removable mounting to a second like adapter.

The second like adapter may also include a hollow body defining an inlet and an outlet for providing a gas flow passage therethrough, a flow restriction for restricting gas flow through the gas flow passage, and an access passage to the gas flow passage, the access passage being provided downstream of the flow restriction. Moreover, the second like adapter may be configured for attachment to a flexible conduit at the outlet.

For the adapter, the hollow body may define an aperture downstream of the inlet for providing the access passage to the gas flow passage.

In particular, the hollow body may include a stepped portion such that a first internal cross-sectional area of the hollow body immediately upstream of the stepped portion is smaller than a second internal cross-sectional area of the hollow body immediately downstream of the stepped portion, and the aperture may be provided in the stepped portion.

The mounting portion have a third internal cross-sectional area which is greater than the first and second internal cross-sectional areas for receiving the second like adapter.

The mounting portion may be configured for sealing engagement with a corresponding hollow body of the second like adapter proximate a corresponding stepped portion of the second like adapter.

The mounting portion may be configured such that a corresponding inlet and access passage aperture of the second like adapter are fully encapsulated in the hollow body of the adapter when the adapter is mounted on the second like adapter.

The flow restriction of the adapter may be different to the flow restriction provided by the second like adapter.

In particular, the flow restriction may be more restrictive than the flow restriction provided by the second like adapter.

Generally, the flow restriction is controlled via the size of the opening at the inlet. The inlet of the adapter may be smaller than the inlet of the second like adapter to provide the more restrictive flow restriction.

In one embodiment, the mounting portion may include a flared portion proximate the outlet of the hollow body for receiving the second like adapter.

In another embodiment, the mounting portion may include a stepped flange proximate the outlet of the hollow body for receiving the second like adapter.

The adapter may further include an engagement portion configured for sealing engagement with a connector associated with a pressure valve.

The hollow body of the adapter may be configured to form a cavity with the connector when the engagement portion is engaged with the connector.

The cavity may be in fluid communication with the gas flow passage via the access passage.

According to another aspect of the invention, there is provided a kit for a respiratory gas delivery system for delivering a gas flow to a patient, the kit including
a flow modifying adapter configured for removable connection to a flow modulator, and
a patient interface configured for providing the gas flow to the patient,
wherein the flow modifying adapter and the patient interface include matching visual indicators.

Any suitable types of visual indicators may be used. The visual indicators may include colour indicators. In particular, a portion of the patient interface may be the same colour as at least a portion of the flow modifying adapter.

The visual indicators may include labels. The labels attached to, and/or integral with the flow modifying adapter and patient interface.

The kit may further include a flexible conduit for connection with the flow modifying adapter.

The kit may further include a humidification chamber for a humidifier.

The kit may further include an inspiration tube for connection with the patient interface.

The kit may further include a filter for the patient interface. The filter may be configured for placement between the patient interface and the inspiration tube.

According to another aspect of the invention, there is provided an adapter comprising
a hollow body defining an inlet and an outlet configured to provide a gas flow passage therethrough,
a flow restriction configured to restrict gas flow through the gas flow passage,
wherein the flow restriction is adjustable.

The adapter may further include an access passage to the gas flow passage, the access passage being provided downstream of the flow restriction.

The adapter may further include a flow restriction adjustment mechanism for adjusting the flow restriction.

Any suitable flow restriction adjustment mechanism may be used. In one embodiment, the flow restriction adjustment mechanism may include a terminal portion movably mounted to an inlet end of the adapter such that relative movement of the terminal portion with respect to the inlet end of the adapter adjusts a size and/or configuration of the flow restriction.

In one embodiment, the terminal portion may include an opening, and a protrusion may be provided at the inlet end of the adapter, wherein relative movement of the terminal portion with respect to the inlet end of the adapter moves the opening relative to the protrusion to adjust the size and configuration of the flow restriction. In an alternative embodiment, the protrusion may be provided on terminal portion, and the opening may be provided at the inlet end of the adapter.

In another embodiment, the terminal portion may include an aperture, and one or more inlet openings may be provided at the inlet end of the adapter, wherein relative movement of the terminal portion with respect to the inlet end of the adapter moves the aperture relative to the one or more inlet openings such that alignment of the aperture with the one or more inlet openings or a portion of an inlet opening adjusts the flow restriction. In an alternative embodiment, the aperture may be provided at the inlet end of the adapter, and the one or more openings for alignment with the aperture may be provided by the terminal portion.

In a further embodiment, the terminal portion may include a cover, and one or more inlet openings may be provided at the inlet end of the adapter, wherein relative movement of the cover with respect to the inlet end of the adapter moves the cover relative to the one or more inlet openings such that an effective size of the one or more inlet openings are adjusted to adjust the flow restriction.

In another embodiment, a plurality of blades may be provided adjacent the inlet end of the adapter, wherein relative movement of the terminal portion with respect to the inlet end of the adapter adjusts the position of the blades to adjust the flow restriction. Each of the blades may be pivotally mounted to the inlet end of the adapter. Pivotal movement of the blades may selectively partially obstruct an opening associated with the inlet end of the adapter to adjust the flow restriction.

In a further embodiment, the flow restriction adjustment mechanism includes an insert receivable by the inlet of the adapter for reducing a size of the flow restriction.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

The disclosure consists in the foregoing and also envisages constructions of which the following gives examples only. Features disclosed herein may be combined into new embodiments of compatible components addressing the same or related inventive concepts.

In order that the invention may be more readily understood and put into practice, one or more preferred embodiments thereof will now be described, by way of example only, with reference to the accompanying drawings.

It will be appreciated that for simplicity and clarity of illustration, where considered appropriate, reference numerals may be repeated among the figures to indicate corresponding or analogous elements.

### BRIEF DESCRIPTION OF THE DRAWINGS

Preferred embodiments of the disclosure will be described by way of example only and with reference to the following drawings.

Specific embodiments and modifications thereof will become apparent to those skilled in the art from the detailed description herein having reference to the figures that follow, of which:

FIG. 1A illustrates a high flow respiratory system.

FIG. 1B is a schematic representation of a pressure valve. (The embodiment in FIG. 1B illustrates a pressure relief valve (PRV) or more specifically a flow-controlled pressure relief valve (FCPRV)).

FIG. 1C is a perspective view of a FCPRV and adapter assembly according to one embodiment.

FIG. 2 is a cross-sectional view of the FCPRV and adapter assembly shown in FIG. 1C.

FIG. 3 is a perspective view of the adapter in the assembly shown in FIG. 2.

FIG. 4 is a cross-sectional view of a FCPRV and adapter assembly according to another embodiment.

FIG. 5 is a perspective view of the adapter in the assembly shown in FIG. 4.

FIG. 6A is a perspective cross-sectional view of a FCPRV and adapter assembly according to another embodiment.

FIG. 6B is a cross-sectional view of a FCPRV and adapter assembly shown in FIG. 6A.

FIG. 7 is a perspective view of the adapter in the assembly of FIGS. 6A and 6B.

FIG. 8 illustrates a tuning routine for a flow-compensated pressure relief valve (FCPRV).

FIG. 9A is a perspective view of an adapter according to a further embodiment.

FIG. 9B is a side elevation view of the adapter of FIG. 9A.

FIG. 9C is a section view of the adapter of FIGS. 9A and 9B, taken through a centreline of the adapter.

FIG. 10 illustrates respiratory system pressure and FCPRV relief pressure response curves with respect to changing input flow rate, wherein the flow rate is the flow rate of gases provided to a patient or from a main outlet of the FCPRV.

FIG. 11A is a perspective view of an adapter (or adapter head portion) according to one embodiment of the invention.

FIG. 11B is a side view of the adapter (or adapter head portion) illustrated in FIG. 11A.

FIG. 11C is a cross sectional view of the adapter (or adapter head portion) as illustrated in FIGS. 11A and 11B.

FIG. 12A is a perspective view of a rigid base portion configured for removable attachment to the adapter head portion shown in FIGS. 11A to 11C.

FIG. 12B is a cross sectional view of the rigid base portion shown in FIG. 12A.

FIGS. 13A and 13B are perspective views of a tethered adapter assembly including a head portion and a base portion according to one embodiment of the invention.

FIG. 14A is a perspective view of an adapter according to a further embodiment of the invention.

FIG. 14B is a cross sectional view of the adapter shown in FIG. 14A.

FIG. 14C is a cross sectional view illustrating the adapter of FIGS. 14A and 14B being removably mounted to a like adapter, the like adapter being an adapter such as the adapter shown in FIGS. 5, 7 and 9A to 9C.

FIG. 15A is a perspective view of an adapter according to a further embodiment of the invention.

FIG. 15B is a cross sectional view of the adapter shown in FIG. 15A.

FIG. 16A is a perspective view of an adapter according to a further embodiment of the invention.

FIG. 16B is a cross sectional view of the adapter shown in FIG. 16A.

FIG. 17 illustrates the system pressure and FCPRV relief pressure response curves with varying flow rate when the system is tuned to adult and paediatric patients.

FIG. 18 illustrates a kit for a respiratory system according to an embodiment of the present invention.

FIG. 19 is a perspective view of an adapter according to one embodiment of the present invention.

FIG. 20 is a perspective view of an adapter according to another embodiment of the present invention.

FIG. 21A to 21E illustrate a flow restriction adjustment mechanism for an adapter according to one embodiment.

FIGS. 22A to 22F illustrate a flow restriction adjustment mechanism for an adapter according to another embodiment.

FIGS. 23A and 23B illustrate a flow restriction adjustment mechanism for an adapter according to a further embodiment.

FIGS. 24A to 24F illustrate a flow restriction adjustment mechanism for an adapter according to yet another embodiment.

FIGS. 25A and 25B illustrate a flow restriction adjustment mechanism for an adapter according to yet another embodiment.

FIGS. 26A to 26F illustrate a flow restriction adjustment mechanism for an adapter according to another embodiment.

FIGS. 27A to 27F illustrate a flow restriction adjustment mechanism for 5an adapter according to another embodiment.

FIGS. 28A to 28H illustrate a flow restriction adjustment mechanism for an adapter according to another embodiment.

FIGS. 29A to 29C illustrate an alternative terminal portion for the flow restriction adjustment mechanism shown in FIGS. 28A to 28H.

FIGS. 30A to 30D illustrate an alignment mechanism for a flow restriction adjustment mechanism.

FIG. 31A illustrates an adaptor having a flow restriction adjustment mechanism according to a further embodiment.

FIGS. 31B to 34C illustrate the flow restriction adjustment mechanism of FIG. 31A.

### DETAILED DESCRIPTION

Various embodiments are described with reference to the figures. Throughout the figures and specification, similar reference numerals may be used to designate the same or similar components, and redundant descriptions thereof may be omitted.

In this specification, “high flow”, “high flows”, “high-flow” or other equivalent terminology means, without limitation, any gas flow with a flow rate that is higher than usual/normal, such as higher than the normal inspiration flow rate of a healthy patient. Alternatively, or additionally, it can be higher than some other threshold flow rate that is relevant to the context-for example, where providing a gas flow to a patient at a flow rate to meet or exceed inspiratory demand, that flow rate might be deemed “high flow” as it is higher than a nominal flow rate that might have otherwise been provided. “High flow” is therefore context dependent, and what constitutes “high flow” depends on many factors such as the health state of the patient, type of procedure/therapy/support being provided, the nature of the patient (big, small, adult, child) and the like. Those skilled in the art know from context what constitutes “high flow”. It is a magnitude of flow rate that is over and above a flow rate that might otherwise be provided.

But, without limitation, some indicative values of high flow can be as follows.

In some configurations, delivery of gases to a patient at a flow rate of greater than or equal to about 5 or 10 litres per minute (5 or 10 LPM or L/min).

In some configurations, delivery of gases to a patient at a flow rate of about 5 or 10 LPM to about 150 LPM, or about 15 LPM to about 95 LPM, or about 20 LPM to about 90 LPM, or about 25 LPM to about 85 LPM, or about 30 LPM to about 80 LPM, or about 35 LPM to about 75 LPM, or about 40 LPM to about 70 LPM, or about 45 LPM to about 65 LPM, or about 50 LPM to about 60 LPM. For example, according to those various embodiments and configurations described herein, a flow rate of gases supplied or provided to an interface via a system or from a flow source or flow modulator, may comprise, but is not limited to, flows of at least about 5, 10, 20, 30, 40, 50, 60, 70, 80, 90, 100, 110, 120, 130, 140, 150 LPM, or more, and useful ranges may be selected to be any of these values (for example, about 20 LPM to about 90 LPM, about 40 LPM to about 70 LPM, about 40 LPM to about 80 LPM, about 50 LPM to about 80 LPM, about 60 LPM to about 80 LPM, about 70 LPM to about 100 LPM, about 70 LPM to about 80 LPM).

In “high flow” the gas delivered will be chosen depending on for example the intended use of a therapy and/or respiratory support. Gases delivered may comprise a percentage of oxygen. In some configurations, the percentage of oxygen in the gases delivered may be about 15% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

In some embodiments, gases delivered may comprise a percentage of carbon dioxide. In some configurations, the percentage of carbon dioxide in the gases delivered may be more than 0%, about 0.3% to about 100%, about 1% to about 100%, about 5% to about 100%, about 10% to about 100%, about 20% to about 100%, or about 30% to about 100%, or about 40% to about 100%, or about 50% to about 100%, or about 60% to about 100%, or about 70% to about 100%, or about 80% to about 100%, or about 90% to about 100%, or about 100%, or 100%.

Flow rates for “high flow” for premature/infants/paediatrics (with body mass in the range of about 1 to about 30 kg) can be different. The flow rate can be set to 0.4-8 L/min/kg with a minimum of about 0.5 L/min and a maximum of about 70 L/min. For patients under 2 kg maximum flow may be set to 8 L/min.

High flow has been found effective in meeting or exceeding the patient's normal real inspiratory flow, to increase oxygenation of the patient and/or reduce the work of breathing. Additionally, high flow therapy and/or respiratory support may generate a flushing effect in the nasopharynx such that the anatomical dead space of the upper airways is flushed by the high incoming gas flows. This creates a reservoir of fresh gas available of each and every breath, while minimising re-breathing of carbon dioxide, nitrogen, etc.

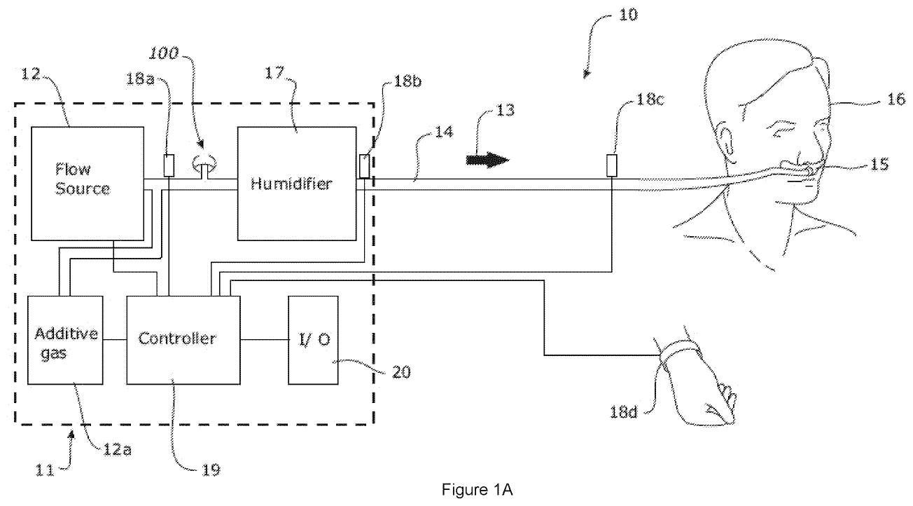

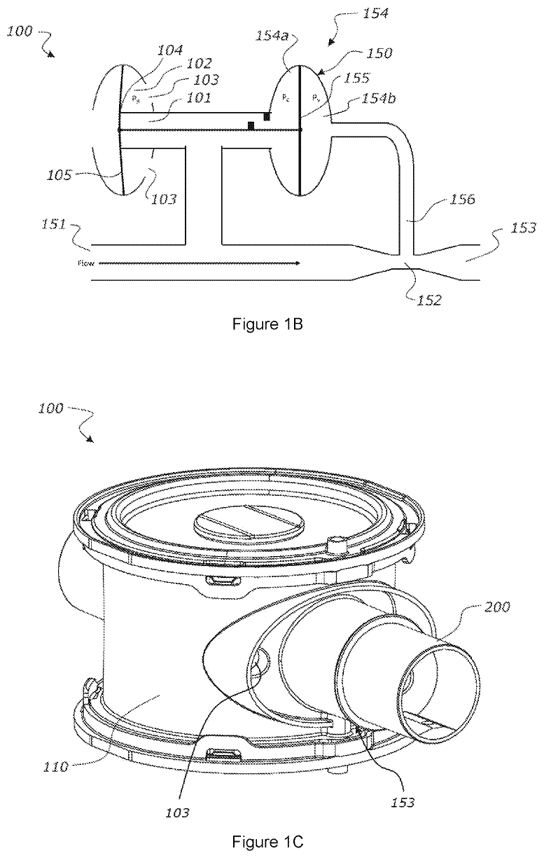

By example, a high flow respiratory system 10 is described below with reference to FIG. 1A. High flow may be used as a means to promote gas exchange and/or respiratory support through the delivery of oxygen and/or other gases, and through the removal of CO₂ from the patient's airways. As mentioned, high flow may be particularly useful prior to, during or after a medical and/or anaesthetic procedure.

When used prior to a medical procedure, high gas flow can pre-load the patient with oxygen (i.e. increase the reservoir of oxygen in the blood) so that their blood oxygen saturation level and volume of oxygen in the lungs is higher than normal in order to provide an oxygen buffer while the patient is in an apnoeic phase during the medical procedure.

A continuous supply of oxygen is important to sustain healthy respiratory function during medical procedures (such as during anaesthesia) where respiratory function might be compromised (e.g. diminishes or stops). When this supply is compromised, conditions such as hypoxia and/or hypercapnia can occur. During medical procedures such as anaesthesia and/or sedation, patient breathing is monitored to detect if spontaneous breathing is diminished or ceases. If oxygen supply and/or CO₂ removal is compromised, the clinician stops the medical procedure and facilitates oxygen supply and/or CO2 removal. This can be achieved for example by manually ventilating the patient for example through bag mask ventilation, or by providing a high flow of gases to the patient's airway using a high flow respiratory system. Further, it will be appreciated that a mask that is used for sedation/ventilation (not necessarily limited to a bag mask) may also be used for pre-oxygenation and also for monitoring patient parameters such as end tidal CO₂, etc.

Further advantages of high gas flow can include that the high gas flow increases pressure in the airways of the patient, thereby providing pressure support that opens airways, the trachea, lungs/alveolar and bronchioles. The opening of these structures enhances oxygenation, and to some extent assists in removal of CO₂ and/or can help support patients with collapsed areas of the lung.

When humidified, the high gas flow can also prevent airways from drying out, mitigating mucociliary damage, reducing risk of infection and reducing risk of laryngospasms and risks associated with airway drying such as nose bleeding, aspiration (as a result of nose bleeding), and airway obstruction, swelling and bleeding. Another advantage of high gas flow is that the flow can clear smoke created during surgery in the air passages. For example, smoke can be created by lasers and/or cauterizing devices.

An adapter according to embodiments described herein is particularly adapted for use in respiratory systems such as continuous positive airway pressure (CPAP) or high flow respiratory gas systems, for example a high flow respiratory support system for use in anaesthesia procedures. Respiratory systems in which the adapter may be particularly useful are CPAP, Bilevel Positive Airway Pressure (BiPAP), high flow respiratory support, varying high flow respiratory support, low flow air, low flow O₂ delivery, bubble CPAP, apnoeic high flow respiratory support (i.e. high flow to anesthetized patients), invasive ventilation and non-invasive ventilation. Further, an adapter as described herein may be useful in systems other than respiratory systems. An adapter according to embodiments described herein is configured for use with a pressure relief or pressure regulating device.

Unless the context suggests otherwise, a flow source provides a flow of gases at a set flow rate. A set flow rate may be a constant flow rate, variable flow rate or may be an oscillating flow rate, for example a sinusoidal flow rate or a flow rate with a step or square wave profile. Unless the context suggests otherwise a pressure source provides a flow of gases at a set pressure. The set pressure may be a constant pressure, variable pressure or may be an oscillating pressure, for example a sinusoidal pressure or a pressure with a step or square wave profile.

With reference to FIG. 1A, the respiratory system 10 may comprise an integrated or separate component-based arrangement, generally shown in the dotted box 11 in FIG. 1A. In some configurations the system 10 could comprise a modular arrangement of components. The respiratory system 10 will be referred to herein as system, but this should not be considered limiting. The system 10 may include a flow source 12, such as an in-wall source of oxygen, an oxygen tank, a blower, a flow therapy apparatus, or any other source of oxygen or other gas. The system 10 may also comprise an additive gas source 12a, comprising one or more other gases that can be combined with the flow source 12. The flow source 12 can provide a pressurised high gas flow 13 that can be delivered to a patient 16 via a delivery conduit 14, and patient interface 15 (such as a nasal cannula). A controller 19 controls the flow source 12 and additive gas source 12a through valves or the like to control flow and other characteristics such as any one or more of pressure, composition, concentration, volume of the high flow gas 13. A humidifier 17 is also optionally provided, which can humidify the gas under control of the controller and control the temperature of the gas. One or more sensors 18a, 18b, 18c, 18d, such as flow, oxygen, pressure, humidity, temperature or other sensors can be placed throughout the system and/or at, on or near the patient 16. The sensors can include a pulse oximeter 18d on the patient for determining the oxygen concentration in the blood.

The controller 19 may be coupled to the flow source 12, the additive gas source 12a, humidifier 17 and sensors 18a-18d. The controller 19 can operate the flow source to provide the delivered flow of gas. It can control the flow, pressure, composition (where more than one gas is being provided), volume and/or other parameters of gas provided by the flow source based on feedback from sensors. The controller 19 can also control any other suitable parameters of the flow source to meet oxygenation requirements. The controller 19 can also control the humidifier 17 based on feedback from the sensors 18a-18d. Using input from the sensors, the controller can determine oxygenation requirements and control parameters of the flow source 12 and/or humidifier 17 as required. An input/output (I/O) interface 20 (such as a display and/or input device) is provided. The input device 20 is for receiving information from a user (e.g. clinician or patient) that can be used for determining oxygenation requirements. In some embodiments, the system may be without a controller and/or I/O interface 20. A medical professional such as a nurse or technician may provide the necessary control function.

The pressure may also be controlled. As noted above, the high gas flow (optionally humidified) can be delivered to the patient 16 via a delivery conduit 14 and the patient interface 15 or ‘interface’, such as a cannula, mask, nasal interface, oral device or combination thereof. In some embodiments, the high gas flow (optionally humidified) can be delivered to the patient 16 for surgical uses, e.g. surgical insufflation. In these embodiments, the ‘interface’ could be a surgical cannula, trocar, or other suitable interface. The patient interface 15 can be substantially sealed, partially sealed or substantially unsealed. A nasal interface as used herein is a device such as a cannula, a nasal mask, nasal pillows, or other type of nasal device or combinations thereof. A nasal interface can also be used in combination with a mask or oral device (such as a tube inserted into the mouth) and/or a mask or oral device (such as a tube inserted into the mouth) that can be detached and/or attached to the nasal interface. A nasal cannula is a nasal interface that includes one or more prongs that are configured to be inserted into a patient's nasal passages. A mask refers to an interface that covers a patient's nasal passages and/or mouth and can also include devices in which portions of the mask that cover the patient's mouth are removable, or other patient interfaces such as laryngeal mask airway or endotracheal tube. A mask also refers to a nasal interface that includes nasal pillows that create a substantial seal with the patient's nostrils. The controller controls the system to provide the required oxygenation.

A system 10 according to embodiments herein includes a pressure valve. A pressure valve may include a pressure regulating device, a pressure relief device, or pressure limiting device. In the specific embodiments described herein, the pressure valve is a flow compensated pressure relief valve or FCPRV 100. The FCPRV 100 may be a valve having features described in WO2018/033863, the entirety of which is hereby incorporated by reference herein. The adapter may be used with other valves and/or devices. The FCPRV may be placed anywhere in the system between the flow source 12 and the patient 16. Preferably, the FCPRV 100 is provided at an outlet of the flow source 12, or between the flow source 12 and the humidifier 17, for example near to an inlet of the humidifier 17. In some embodiments, the FCPRV 100 may be provided at an outlet of the humidifier 17 and/or an inlet to the conduit 14, or at any point along the conduit 14 through a suitable housing or coupling device. The FCPRV 100 may be located anywhere in the system, for example the FCPRV could be part of the patient interface 15.

A FCPRV 100 according to the present disclosure relieves pressure at an approximately consistent pressure across a given range of flow rates. The FCPRV 100 may be used to provide an upper limit for patient safety, and/or to prevent damage to system components caused by overpressure. For example, an occlusion in the system may cause a substantial back pressure in the system upstream of the occlusion, and the FCPRV may operate to ensure the back pressure does not increase above a limit to protect the patient and/or system components from damage. A blockage in the patient's nares or exhaling conduit can result in an increased patient pressure. An occlusion in the system may be caused by, for example, inadvertent folding or crushing of the conduit 14, or may be caused deliberately, for example by occluding the conduit 14 (e.g. by pinching a portion of the conduit closed) to prevent a flow of gases from reaching the patient.

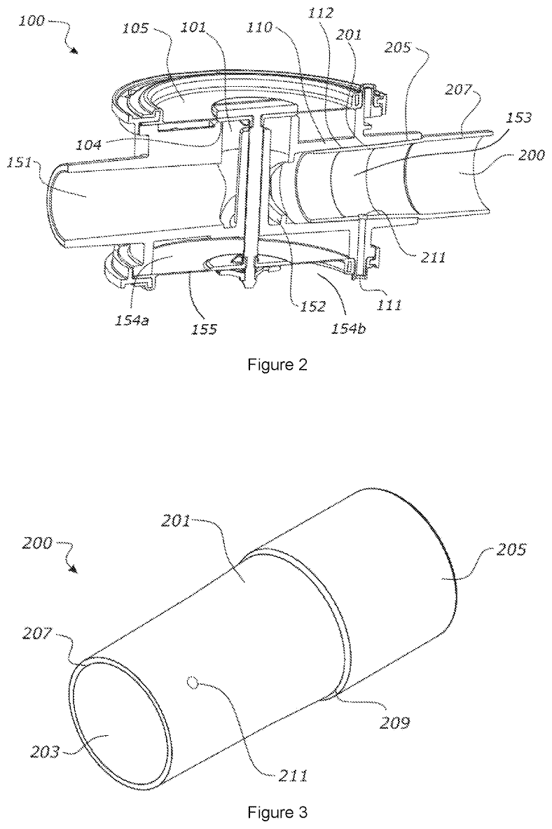

FIGS. 1C and 2 show one embodiment of a FCPRV 100, which is illustrated schematically in FIG. 1B. The FCPRV 100 comprises an inlet 101, an outlet chamber 102 with an outlet 103, a valve seat 104 between the inlet 101 and the outlet chamber 102, and a valve member 105 biased to seal against the valve seat 104. The valve member 105 is adapted to displace from the valve seat by pressure P_{c} at the FCPRV inlet 101 increasing above a pressure threshold. The pressure P_{c} acts on the valve member 105 to force the member away from the valve seat 104 once the pressure P_{c} reaches or exceeds the threshold. As the valve member 105 displaces from the valve seat 104, a flow of gases flows from the inlet 101 into the outlet chamber 102, and then from the outlet chamber 102 via the outlet 103 to to ambient pressure/atmospheric pressure. The outlet from the chamber is configured so that the flow of gases through the outlet causes a (back) pressure Pb in the outlet chamber that acts on the valve member 105 to further displace the valve member 105 from the valve seat 104. As the valve member 105 is further displaced from the valve seat 104, a gap between the valve member 105 and valve seat 104 increases.

The FCPRV 100 further comprises a sensing mechanism 150 to dynamically adjust the pressure threshold at which the FCPRV 100 vents pressure based on the flow rate and/or pressure of the gases or a portion thereof, passing through the FCPRV or through the respiratory system. In certain embodiments, the FCPRV 100 comprises a sensing mechanism 150 to dynamically adjust the pressure threshold at which it vents pressure based on the flow rate of the gases or a portion thereof, passing through the FCPRV or through the respiratory system. In certain embodiments, the FCPRV 100 comprises a sensing mechanism 150 to dynamically adjust the pressure threshold at which it vents pressure based on the pressure of the gases or a portion thereof, passing through the FCPRV or through the respiratory system. An adapter 200 according to embodiments described herein can be used with the FCPRV 100.

Referring to FIGS. 1B and 2, features and functionality of the FCPRV will now be described. The FCPRV 100 comprises a body 110 defining a main inlet 151 and a main outlet 153. In the illustrated embodiment, the sensing mechanism 150 includes a flow restriction or flow constriction 152 between the main inlet 151 and main outlet 153 of the FCPRV. The main inlet 151 and/or main outlet 153 are preferably integral with or defined by the FCPRV body 110. In the embodiment of FIGS. 1B and 2, the flow restriction 152 is part of the FCPRV body. In later described embodiments, the flow restriction is part of the adapter. For ease of reference, the term ‘flow restriction’ may be used herein to describe both a flow restriction such as an orifice plate and a flow constriction such as used in a venturi. In operation, the flow of gases in a respiratory system flow through the FCPRV 100 from the main inlet 151 to the main outlet 153. The sensing mechanism 150 senses the flow rate/pressure of gases flowing to the patient at or downstream of the flow /striction/ constriction. In the embodiment shown, the inlet 101 is between the main inlet 151 and main outlet 153, and the flow restriction/constriction is downstream of the inlet 101, but upstream of the main outlet 153. The sensing mechanism 150 senses the flow rate and/or pressure of gases flowing to the patient at or through the main outlet 153 of the valve.

The sensing mechanism 150 also includes a sensing chamber 154, and a sensing member 155 located in the sensing chamber 154. The sensing member 155 divides the sensing chamber 154 into a first chamber 154a and a second chamber 154b. The first chamber 154a is in fluid communication with the flow of gases upstream of the flow restriction 152, e.g. the first chamber 154a is in fluid communication with the main inlet 151 and the valve inlet 101 upstream of the restriction 152. The second chamber 154b is in fluid communication with the flow of gases at the flow constriction 152 or downstream of the flow restriction 152. In some embodiments, the device comprises a flow constriction configured as a venturi, with the second chamber 154b in fluid communication with the constriction via a pressure ‘tap’ or communication line 156 (FIG. 1B). However, in an alternative configuration the device may comprise a flow restriction 152, e.g. an orifice plate, and the first and second chambers may tap off either side of the orifice plate, for example via pressure ‘tap’ or communication line 111 shown in FIG. 2. A pressure differential may be generated in any other suitable way, for example by a permeable membrane or a filter with a known pressure drop (a flow restriction).

A resulting pressure drop caused by the flow of gases that pass from the main inlet 151 to the main outlet 153 of the device, through the restriction 152 is therefore sensed by the sensing member 155 located within the sensing chamber 154.

In order to increase the flow rate through the respiratory system 100, the pressure provided by the flow source 12 is increased, increasing the pressure at the main inlet 151 and also in the first chamber 154a of the sensing chamber 154. As the flow rate through the FCPRV increases, a larger pressure drop is created by the restriction 152 due to an increased velocity of the gases passing through the restriction 152, and the pressure Pᵥ in the second chamber 154b of the sensing chamber 154 decreases. Thus, an increasing flow rate through the FCPRV 100 from the main inlet 151 to the main outlet 153 results in an increasing differential pressure across the sensing member 155, with the first chamber 154a being a high (higher) pressure side of the sensing chamber 154 and the second chamber 154b being a low (lower) pressure side of the sensing chamber 154. This causes the sensing member 155 to move towards the low-pressure side of the sensing chamber 154, away from the valve member 105.

The sensing member 155 is mechanically coupled to the valve member 105 of the FCPRV 100, so that as the sensing member 155 moves towards the lower pressure side of the sensing chamber 154, the sensing member 155 pulls or biases the valve member 105 of the FCPRV against the valve seat 104. For a given flow rate setting, a higher flow rate causes a higher differential pressure across the sensing member 155, biasing the valve member 105 further towards the valve seat 104. This causes the pressure relief threshold for the FCPRV 100 to increase. If a flow restriction (e.g. squashed conduit 14 or blockage in patient's nare) is introduced, the flow source 12 (rapidly) adjusts to increase pressure in the system to maintain the flow rate at a desired level. If the system pressure required to maintain the desired flow rate is above the relief pressure, the FCPRV begins to vent, with a portion of the flow provided to the main inlet 151 venting via the FCPRV valve member 105, and a portion of the flow passing through the restriction 152 and from the main outlet 153. The flow source 12 maintains a set flow rate to the main inlet 151 of the FCPRV 100. Thus, as the FCPRV begins to vent, the flow rate through the constriction or restriction 152 decreases, and the pressure differential acting on the sensing member 155 decreases. This causes the bias provided by the sensing member 155 to the valve member 105 to decrease, and therefore the pressure relief threshold for the FCPRV 100 to decrease. In an ideal situation, an equilibrium state will be reached, whereby the patient receives as much flow as possible without exceeding the pressure relief threshold, or without exceeding a maximum delivered pressure at the patient interface.

If the flow restriction completely (or substantially completely) blocks the system, for example a conduit 14 is completely occluded (complete crushed or pinched closed) or a patient's nare is completely blocked, all or substantially all flow delivered to the main inlet 151 of the FCPRV 100 is vented via the valve member 105.

FIG. 1B shows a body that provides or forms the outlet chamber 102 and the first chamber 154a of the sensing chamber 154. Those features are not shown in the other figures, but it will be appreciated that any of the embodiments of the FCPRV or adapter described herein may be used with a valve body having those features.

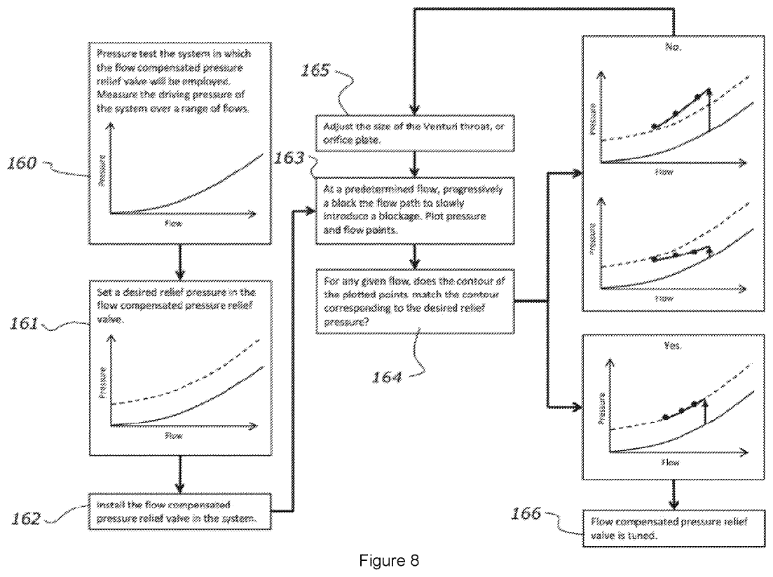

FIG. 8 illustrates a tuning method for tuning the FCPRV 100. At step 160 the system 10 is pressure tested to determine a system flow (e.g. flow delivered to the patient) versus the overall pressure drop response curve for the system 10. In step 161 a desired relief pressure v flow curve is determined, for example by adding an offset pressure to the system pressure v flow curve. At step 162, the FCPRV 100 is installed in the system 10. At step 163, a flow restriction is then progressively added to the system downstream of the FCPRV 100, and the resulting relief pressure for a range of flow rates is determined to create a curve of measured pressure relief vs flow rate. At step 164, the actual pressure relief v flow curve is compared to the desired curve. At step 165, if the actual curve does not match the desired curve, the size of the flow restriction (Venturi throat or orifice) is adjusted and steps 163 and 164 are repeated again, until the desired pressure relief characteristic is achieved, at which point at step 166 the FCPRV 100 has been successfully tuned.

As illustrated in FIG. 8, a gradient of the relief pressure response curve can be adjusted by altering the size of the flow restriction. A greater gradient indicates a higher rate of change in the FCPRV relief pressure with respect to increasing flow rate. By altering the gradient, the flow and pressure characteristics of a specific respiratory system can be tuned for optimal performance for a particular patient population. Accordingly, in some embodiments, the gradient can be altered for optimal performance in a particular respiratory system by adjusting the size of the flow restriction on the adapter.

Alternatively, or additionally the vent pressure threshold may be adjusted by adjusting any one or more of the other features of the FCPRV. For example, the tension in the valve membrane 105 may be adjusted by for example adjusting the relative position of the valve inlet 101 to the valve member 105, or the size of the vent outlet 103. In the FCPRV 100, the size of the vent outlets determines the shape of the pressure relief valve relief pressure v flow curve and therefore the vent pressure threshold over a range of flow rates. When the system is completely blocked/occluded, the sensing member may provide some additional bias to the valve member 105. Also, the biasing force provided to the valve member 105 by the sensing member 155 may be adjustable. For example, the length of the mechanical link 157 between the sensing and valve members may be adjustable, a shorter length link increasing the biasing force and therefore the vent pressure.

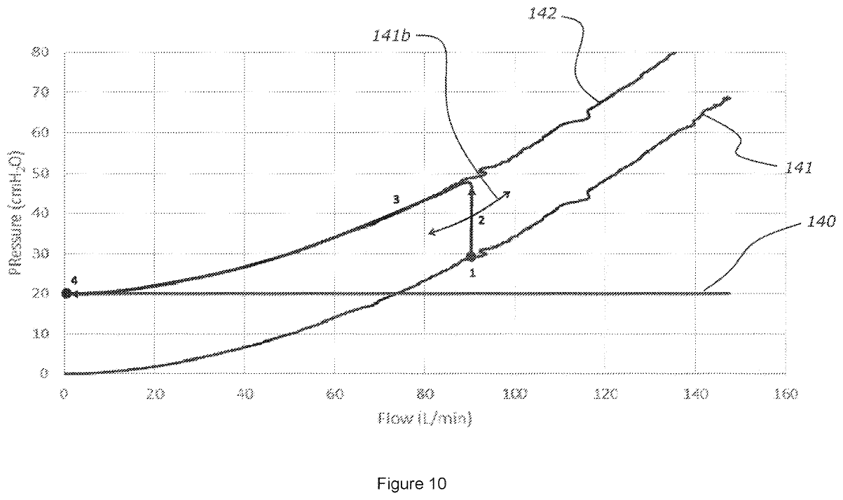

Four stages of operation of the FCPRV 100 will now be described with reference to FIG. 10. In stage 1, indicated by point 1 on curve 141 indicating pressure in the respiratory system 10, the respiratory system 10 is providing a flow of gases to a patient 16. All (or substantially all) of the flow provided from the flow source 12 to the main inlet 151 of the FCPRV 100 is delivered to the system 10 from the main outlet 153 of the FCPRV 100. At point 1 a very low or ambient pressure is being delivered to the patient 16 since all pressure is being dropped through the system 10. As the flow rate delivered to the patient 16 is adjusted up and down, for example by a user, the pressure relief threshold of the FCPRV 100 varies along the relief pressure v flow curve 142—as the flow rate to the patient increases, the increasing differential pressure sensed by the sensing member 155 acts on the valve member 105 to increase the FCPRV vent threshold pressure as indicated by curve 142.

For a given flow rate setting (90 L/min in FIG. 10), in stage 2, in a situation where a flow restriction is introduced to the system 10, for example by the partial occlusion of a patient inspiratory conduit 14, or a squashed nasal prong of a nasal cannula patient interface 15, or more importantly a blockage at the patient, for example between a nasal prong and the patient's nares, the flow rate may instantaneously decrease. However, in a set flow system, the flow source 12 (rapidly) adjusts to increase pressure in the system to maintain the flow rate at a desired level. The drop in flow rate and then increase in pressure by the flow source response to maintain a set flow rate to the FCPRV may occur essentially instantaneously, i.e. very quickly, and is therefore negligible. As the flow is maintained, the differential pressure caused by the flow constriction or restriction 152 of the FCPRV remains constant, the bias provided to the valve member 105 by the sensing member 155 remains constant, and therefore the relief pressure threshold for the FCPRV 100 remains constant. However, as the system pressure has increased (for example due to an increased pressure in the patient's airway/nares), the pressure Pc acting on the valve member 105 (and the sensing member 155 on the high-pressure side of the sensing chamber 154) is increased towards the relief pressure of the FCPRV 100. This situation is represented by the vertical arrow 2 in FIG. 10. If a partial occlusion was held and an equilibrium condition reached, a higher system pressure v flow curve indicated by curve 141b in FIG. 10 would result, with a smaller offset between the higher system pressure v flow curve 141b and the FCPRV relief pressure v flow curve 142. For example, for a partial blockage between a nasal prong and a patient's nare that results in an increased system pressure 141b, the pressure generated in the patient's nare is the offset between curve 141b and curve 141. In stage 3, the introduced flow restriction (e.g. squashed conduit 14 or blockage in patient's nare) is increased to a level whereby the system pressure required to maintain the desired flow rate is above the relief pressure 142 of the flow compensated relief valve for the given flow rate (about 90 L/min in FIG. 10). As the system pressure at the FCPRV (e.g. P_{c}) exceeds the flow compensated relief pressure 142, the FCPRV 100 begins to vent, with a portion of the flow provided to the main inlet 151 venting via the FCPRV 100 and a portion of the flow passing through the restriction 152 and from the main outlet 153. The flow source maintains a set flow rate to the main inlet of the FCPRV. Thus, as the FCPRV 100 begins to vent, the flow rate through the constriction or restriction 152 decreases, and the pressure differential acting on the sensing member 155 decreases. This causes the bias provided by the sensing member 155 to the valve member 105 via the mechanical link 157 to decrease, and therefore the pressure relief threshold for the FCPRV 100 to decrease. This situation is represented by arrow 3 on the pressure relief v flow curve 142 in FIG. 10. In an ideal situation, an equilibrium state will be reached, whereby the patient receives as much flow as possible without exceeding the pressure relief threshold, or without exceeding a maximum delivered pressure at the patient interface.

In stage 4, indicated by point 4 in FIG. 10, the flow restriction introduced to the system may completely (or substantially completely) block the system, for example a conduit 14 is completely occluded (complete crushed or pinched closed) or a patient's nare is completely blocked. All flow delivered to the main inlet 151 is vented via the FCPRV 100. As there is no flow through the device 100 from the inlet 151 to the outlet 153, and therefore no flow through the constriction/restriction 152, the pressures in the first and second chambers 154a, 154b are equal and the sensing member 155 provides a minimum bias to the valve member 105. Changes in pressure Pc does not change the pressure differential across the sensing membrane 155.

Thus, in a situation whereby a patient's nares are blocked, the maximum pressure that the patient can receive is the offset between the relief pressure curve 142 and the system pressure drop curve 141, protecting the patient against overpressure. For example, in FIG. 10 this maximum patient pressure is 20 cmH₂O. Thus, the FCPRV provides a venting pressure threshold that is dependent on flowrate yet simultaneously sets an upper pressure limit that the patient will receive. The FCPRV must be capable of venting the maximum flow rate provided by the flow source 12, to ensure the FCPRV can vent the system along curve 142 back to zero flow to the patient, otherwise a higher patient pressure than the indicated offset pressure may eventuate.

The above-described operation of the FCPRV is for a system providing a flow of gases to a user via an unsealed or non-sealing patient interface, such as a nasal cannula that does not seal with the patient's nares. The gases source in such a system may be a compressed gas tank or a hospital wall flow meter supply, or a blower capable of providing sufficient flow rate, or other suitable source that has the ability to provide a rapid response to variation in system resistance to maintain a set flow to the system. A system including a flow meter 12 providing a set flow rate of gases to a patient via a FCPRV, humidifier 17, filter and an unsealed nasal cannula 15 is illustrated in FIG. 1A. Such a system is particularly adapted for providing nasal high flow therapy.

FIGS. 2 and 3 show the FCPRV 100 with one embodiment of an adapter 200 for coupling the FCPRV to a conduit for the supply of gas to a patient. The embodiment of the adapter 200 shown in FIG. 2 is a single part. The adapter 200 is a male adapter. The adapter 200 is configured for use with a connector associated with the outlet of the FCPRV 100 (also referred to herein as the ‘FCPRV connector’), which is a female connector provided by the FCPRV 100. An example of a female connector is the valve body 110 at the outlet 205, as shown in FIGS. 1C and 2.

Referring to FIGS. 2 and 3, features of one embodiment of the adapter 200 will now be described. The adapter 200 has a hollow body with an inlet 203 and an outlet 205. The inlet 203 and outlet 205 define a gas flow passage therebetween. In some embodiments, the gas flow passage is or comprises a pressure line. The gas flow passage is defined at least in part by a wall 207 of the adapter 200. The wall 207 provides an adapter that is a generally tubular component having a generally cylindrical body that may be tapered and/or vary in its cross-sectional area along the length of the adapter 200. In other embodiments, the adapter 200 comprises other cross-sectional shapes, e.g., elliptical, oval, obround, square and rectangle.

The body of the adapter 200 has an overlap portion 201 that is configured to overlap with a portion of the connector associated with the FCPRV 100, when connected thereto. The adapter 200 has an access passage, an access aperture, or an access hole, extending through the overlap portion 201 to the gas flow passage. The access passage fluidly communicates with the gas flow passage of the adapter to enable sensing of the pressure in the gas flow passage. In this embodiment, the access passage comprises an aperture 211. In the embodiment shown in FIGS. 2 and 3, the aperture extends through the wall 207 of the adapter 200. This embodiment has a single aperture 211. The aperture 211 has a similar size and shape to that of the bleed line 111. In alternative embodiments, there may be more than one aperture 211 extending through the wall 207. The adapter 200 may have alignment features (not shown) to guide the adapter towards the correct alignment position to ensure the aperture 211 is aligned with the bleed line 111. Examples of alignment features include two dimensional features such as text, symbols, and arrows. Other examples of alignment features include three dimensional features such as complementary protrusions and recesses. In various embodiments, the adapter 200 may have one or more alignment positions with respect to the main outlet 153 of the FCPRV 100, to facilitate obtaining or not obtaining a flow and/or pressure compensated response from valve 100 or not obtaining any pressure relief from valve 100. In a first configuration, the aperture 211 is not aligned with the bleed line 111 of the sensing mechanism and so there is no fluid communication between the gas flow passage through the adapter 200 and the sensing chamber 154 via the access passage 211 such that the valve 100 will not provide any pressure relief functionality but will still allow gases to flow through the flow passage between main inlet 151 and main outlet 153. In such a configuration, the valve does not function as a pressure relief valve. In a second configuration, the aperture 211 is aligned with the bleed line 111 such that there is fluid communication between the gas flow passage through the adapter 200 and the sensing chamber 154 via the bleed line 111 of the sensing mechanism and the access passage 211. The FCPRV 100 thereby functions as a flow and/or pressure compensated pressure relief valve as described above.

External features of the adapter 200 preferably seal with internal features of the connector, for example, the main outlet 153 of the valve body 110. In this embodiment, a portion of the exterior surface of the adapter 200 is tapered. The surface is tapered inwardly towards the terminal end (inlet 203) of the adapter 200. The taper is preferably a constant taper. The adapter body tapers outwardly from the terminal end, from a smaller diameter to a larger diameter. In other embodiments, the adapter 200 may have a constant diameter.

The main outlet 153 of the valve body 110 has a complementary size and taper such that the components preferably seal when assembled. Further embodiments are described below in which the connection between the main outlet 153 and the adapter create the effect of a low pass filter between the flow passage through the adapter and the sensing mechanism. In this embodiment, there is no low pass filter effect because a cavity is not formed between the walls of the main outlet 153 and the adapter 200, where the cavity is in fluid communication with the gas flow passage and bleed line 111.

The adapter 200 may comprise a stop. In the embodiment shown, the stop is a shoulder 209. The shoulder 209 is integral with the adapter body. The shoulder 209 is positioned to abut the terminal end of the FCPRV outlet 153/connector when the adapter 200 is assembled with the FCPRV body, thereby to prevent, or at least substantially inhibit the adapter 200 being over-inserted into the connector.

The adapter 200 may further comprise an engagement mechanism configured to couple the adapter to the FCPRV 100. In the embodiment shown in FIGS. 2 and 3, the fit between the adapter 200 and the main outlet 153 of the valve body 110 acts as an engagement mechanism. That is, the adapter 200 is retained in place due to frictional forces between the internal walls of the connector/main outlet 153 and the external surface of the adapter 200.

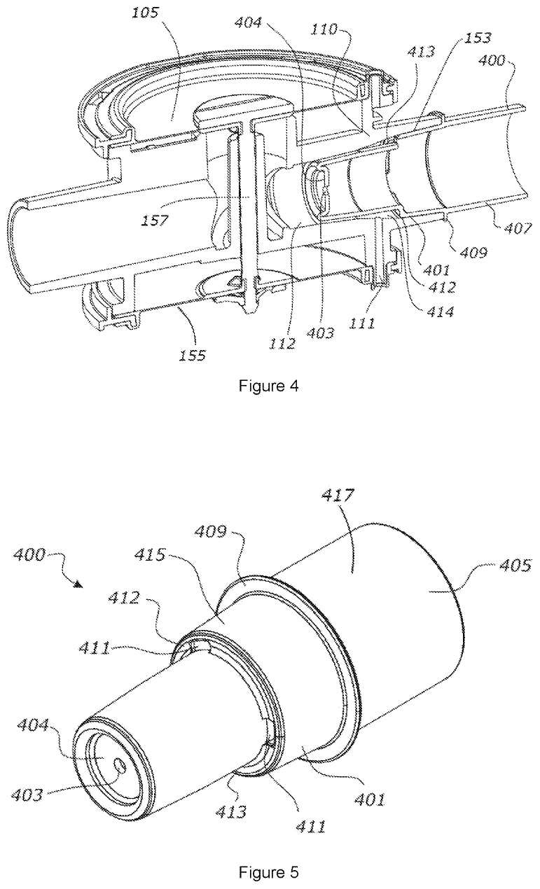

Another (second) embodiment of the adapter will now be described with reference to FIGS. 4 and 5. The adapter 400 has the same features and functionality of the first adapter 200, unless described below. Like numbers are used to indicate like parts with the addition of 200.

In this embodiment, the adapter has a cavity forming portion 413 and a sealing mechanism 415. When the adapter 400 and the valve 100 are assembled, the sealing mechanism 415 substantially pneumatically seals the adapter 400 and the main outlet 153 of the valve body 110. The cavity forming portion 413 and the main outlet 153 of the valve body 110 form a cavity.

The cavity forming portion 413 is a recess or change in a surface of the adapter body that faces away from the gas flow passage. The exterior surface of the cavity forming portion has a shape that is not complementary to the internal surface of the main outlet 153 of the FCPRV 100, such that when assembled, the surfaces may be configured (e.g., having converging, diverging and/or parallel portions) to form a cavity 414. In the embodiment, a recess is provided by a stepped portion of the adapter outer surface, while the main outlet 153 of the valve body 110 does not have a complementary shape. Rather, the main outlet 153 of the valve body 110 has a gradual taper such that when assembled, the adapter 400 and main outlet 153 define a cavity 414 therebetween. In other configurations, the main outlet 153 of the valve body 110 may not have a taper. The cavity 414 is defined by an internal surface of the main outlet 153 of the valve body 110 and the cavity forming portion 413, when the adapter 400 is coupled to the main outlet 153. In addition to having a stepped portion, the cavity forming portion 413 comprises an arcuate (includes but is not limited to curved) surface, preferably a radial surface. The arcuate surface is defined by the cylindrical adapter body.

When formed, the cavity 414 is in fluid communication with the bleed line 111. The formed cavity 414 is in fluid communication with the gas flow passage via the access passage 411. The access passage comprises one or more apertures 411. This arrangement allows the communication of pressure in the gas flow passage through the apertures 411 into the cavity 414 and then subsequently into the bleed line 111 and the second chamber 154b, which can create a pressure differential across the sensing member 155 in the sensing chamber 154 so that the FCPRV 100 can function as described above.

In the embodiment shown, the cavity forming portion 413 has a longitudinal dimension along a longitudinal axis that is substantially parallel to a direction of gas flow in the gas flow passage. In an alternative embodiment, the cavity forming portion 413 may not be substantially parallel to a direction of gas flow in the gas flow passage. In this embodiment, the one or more apertures 411 are arranged substantially parallel or substantially perpendicular to a direction of gas flow in the gas flow passage. The location and formation of the cavity 414 in relation to the bleed line 111 or opening of the bleed line 111 can vary, provided it is in fluid communication with the bleed line 111 via the apertures 411.

In this embodiment, apertures 411 are arranged on the stepped portion/shoulder 412 formed between the cavity forming portion 413 and the sealing portion 415. This embodiment includes three apertures 411 that are radially arranged about the gas flow passage. There may be more apertures 411, for example, four or five apertures 411. There may be fewer apertures 411, for example, one or two apertures.

At least one aperture 411 may be in fluid communication with the gas flow passage via another aperture, the apertures being connected and in fluid communication by a channel (for example, a port in the wall of the adapter that allows for downstream sampling).

FIGS. 4 and 5 show the inlet end (terminal end) of the adapter includes a wall 404 having an inlet aperture 403 that provides a flow restriction or an additional flow restriction. The inlet aperture 403 is also the inlet of the adapter 400. The wall 404 is spaced inwardly from the end of the adapter, forming a recess. The wall 404 is located slightly inwardly, spaced from the terminal end, which increases the stiffness of the terminal end. The inlet aperture 403 is a tuning aperture in conjunction with a radial clearance, as described below. In an alternative embodiment, the wall 404 and inlet aperture 403 may be arranged directly at the terminal end of the adapter 400. In another alternative embodiment, the aperture 403 may be absent, that is, the wall 404 is a continuous wall. In such an embodiment, all of the gas flows through the access passage apertures 411.

The sealing mechanism 415 is configured to form a first seal with a portion of the main outlet 153 of the valve body 110. The sealing mechanism may comprise one or more of sealing mechanisms known in the art, e.g., a face seal, an O-ring, a lip seal, a wiper seal, or a sealing surface. In the embodiment shown in FIGS. 4 and 5, the sealing mechanism is a sealing surface 415.

The cavity 414 is upstream of the sealing mechanism. In this case, the seal comprises an outer seal, that is, a seal that is proximate the terminal end of the main outlet 153 and/or proximate the collar 409 of the adapter of FIG. 4, for the valve to function. FIG. 4 shows an example of an embodiment with this outer seal, as the sealing surface, where the outer seal is formed by engagement or interaction of a portion of the exterior wall of the adapter 400 with a portion of the interior wall of the main outlet 153. It should be noted that other embodiments described with more than one seal could also be implemented with a single sealing surface. An outer seal can be defined as a seal that is downstream of the bleed line 111 and cavity 414 that is formed.

By providing a valve body 110, and a separate adapter 400, it is possible for the features of the valve body 110 to be set or fixed, while the pressure relief characteristics can be readily tuned by altering and/or adjusting the features of the adapter or changing the adapter used. Rather than providing a large number of different FCPRVs, it is possible to provide one design of a valve body and a variety of different adapters. Each adapter can be specifically tuned to provide the desired features, functionality and/or pressure relief characteristics, for example sealed and unsealed respiratory systems, and differentially sized patient interfaces (e.g. nasal cannulas), and different types of patients (e.g., adult patients or paediatric patients), as different flow rates and different system components (e.g. different patient interfaces) are typically required for patient populations. For example, at step 165 of the tuning process illustrated in FIG. 8, the size of the flow restriction can be adjusted by changing the adapter to one having a different sized inlet 403.

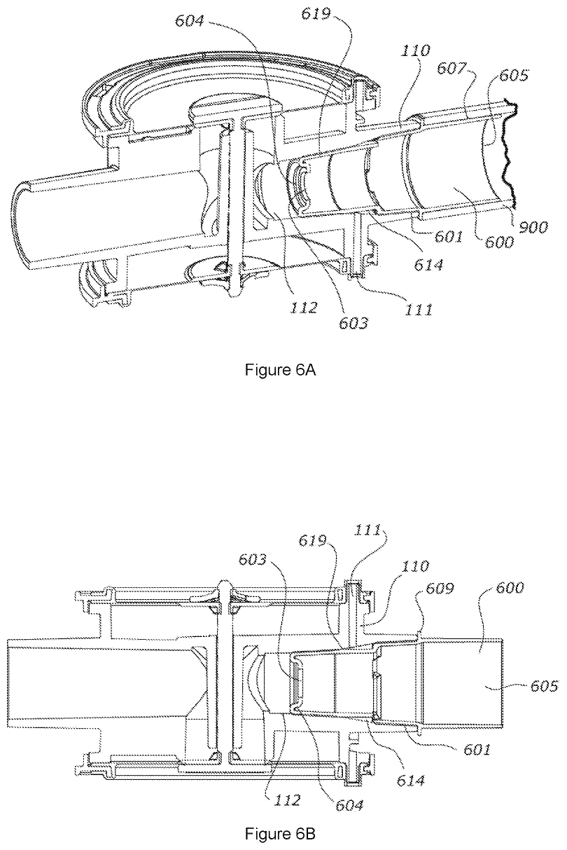

As shown in FIG. 6, in some embodiments an additional inner seal 619 may be present. An inner seal 619, described further below, comprises a seal that is upstream of the bleed line 111 and cavity 614 that is formed. This inner seal may be proximate to the centre of the FCPRV when the adapter 600 is engaged with the main outlet 153.

In alternative embodiments, other configurations of the adapter and the valve body 110 may be used to form the cavity 414, 614. For example, the main outlet 153 of the valve body 110 may have a stepped portion and the adapter may have a gradual taper. In another alternative embodiment, the main outlet 153 of the valve may have a taper and the adapter may have a different taper. In another alternative embodiment, the main outlet 153 of the valve body 110 may have a stepped portion change and the adapter 400 may have a stepped portion change, where the stepped portion changes are offset in a direction that is parallel to the direction of gas flow, forming a cavity. Further, the shape of the adapter 400 and the shape of the main outlet 153 of the valve body 110 or other parts of the valve, together with the configuration of those components when assembled may be chosen or designed such that there is a tolerance and the components do not have to line up exactly to form a suitable cavity.

In the embodiment 400 of FIGS. 4 and 5, a radial clearance, at high fluid velocity, occurs. Flow accelerates through the apertures 411 and creates low pressure areas. In this embodiment there is an annular cavity 414 created that is sealed only at one end (outer seal). The size of the annular cavity 414 between the adapter 400 and the internal wall of the main outlet 153 of the valve body 110 where there is no seal will have to be taken into account so that venting occurs as desired. As the cavity is only sealed at one end, the other end is in fluid communication with the gas flow passage, which may make tuning of the valve more difficult. The valve tuning has to take into account the leak flow into the cavity 414 which can affect the pressure differential across the sensing member 155 in the sensing chamber 154. Tuning the valve involves adjusting the size of the tuning aperture 403 or changing the diameter of the main outlet 153 and/or cavity forming portion 413 to change the size of the radial clearance to achieve a desired response. Changing the radial clearance will adjust the flow velocity. The relative sizes of the aperture 403 and the radial clearance will change the ratio of flow taking each path. This may be achieved by substituting a different adapter with differently dimensioned inlet aperture 403, outlet 153, and/or cavity forming portion 413.

The adapter may comprise a stop. In the embodiment shown, the stop is a collar 409. In the embodiment shown, the collar 409 is an annular collar. In alternative embodiments, the stop may be another feature that comprises the collar 409. The collar 409 is integral with the adapter body. In an alternative embodiment, the collar 409 may be a separate component that is assembled with the adapter body. A surface of the collar 409 may be configured to form a face seal with a surface of the FCPRV connector. In other configurations, the collar 409 may replace or aid the sealing mechanism 415. The collar 409 prevents, or at least substantially inhibits the adapter 400 being over-inserted into the connector of the FCPRV.

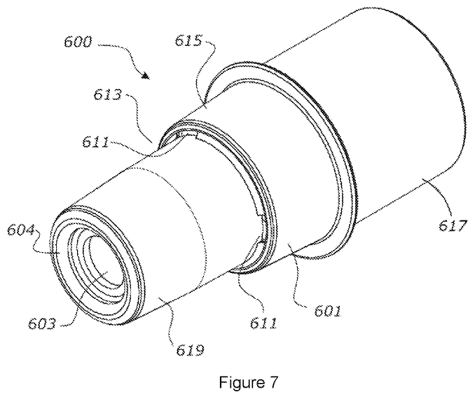

Another (third) embodiment of the adapter will now be described with reference to FIGS. 6 and 7. The adapter 600 has the same features and functionality of the second adapter 400, unless described below. Like numbers are used to indicate like parts with the addition of 200.

In this embodiment, there is a first sealing mechanism 615 and a second sealing mechanism 619. Embodiments of the adapter having two sealing mechanisms facilitate tuning of the response of the FCPRV. The cavity forming portion 613 is between the first sealing mechanism 615 and the second sealing mechanism 619. The access passage is in fluid communication with the cavity 614. The access passage is also positioned between the first sealing mechanism 613 and the second sealing mechanism 615. In the embodiment of FIGS. 6 and 7, the cavity 614 that is formed between the first sealing mechanism 615 and the second sealing mechanism 619, when the adapter 600 is coupled to the main outlet 153, is an annular cavity. That is because the main outlet 153 of the valve body 110 has a radial bore and the adapter 600 has a radial outer surface.

In the embodiment shown in FIGS. 6A, 6B and 7, the second sealing mechanism 619 is a sealing surface. The first sealing mechanism 615 and the second sealing mechanism 619 are formed by the interference/friction fit of the external surfaces of the adapter 600 and the complementary inner surface(s) of the main outlet 153 of the valve body 110 as shown. However, many other methods may be used to create seals and form a cavity. For example, O-rings, wiper seals, adhesives, foams or lip seals may be used at different locations on an adapter and seal with internal or external surfaces of the female connector (valve body 110) to form the cavity 614. Further, an internal interference fit may be used for one seal in conjunction with retention features such as a tab and clip or other external sealing method on the outside of the valve/connection assembly to create a cavity.

FIGS. 6A, 6B and 7 show a preferred assembly, in which the overlap portion 601 includes the first sealing mechanism 615. The overlap portion 601 also comprises the second sealing mechanism 619. In an alternative embodiment, the overlap portion 601 may comprise only one of the sealing mechanisms.

FIG. 6B shows the main outlet 153 of the valve body 110 has an internal, gradually tapered bore. This internal bore of the main outlet 153 of the valve body 110 has non-standard diameters. This is to avoid the connection of incorrect adapters with the main outlet 153. In this embodiment, the flow restriction is provided by the aperture 603 at the inlet to the adapter (rather than by the valve body). If an incorrect adapter is made that fits into the main outlet 153, the valve is unlikely to operate as a flow and/or pressure compensated valve or a valve that provides pressure relief because the valve and adapter would not have a flow restriction and/or an access passage with the main gas flow path to achieve the flow rate and/or pressure sensing as described in relation to the embodiments of the valve and adapter. In this case, if an incorrect adapter that does not have a flow restriction, but which provides fluid communication between the second sensing chamber and the main gas flow passage between main inlet 151 and main outlet 153, (e.g. via the communication line 111), is used with the FCPRV body, the pressure response of the valve 100 will match the response observed when the outlet 153 of the valve 153 is blocked and gases are venting from the valve. That could include a substantially flat response of, for example, 20 cm H₂O. If an incorrect adapter that does not provide fluid communication between the second sensing chamber and the main gas flow passage between main inlet 151 and main outlet 153 (i.e., the communication line 111 is blocked), is used with the FCPRV body, the valve 100 will not provide any pressure relief during use but gases are still able to flow through the main flow passage. As a result, the respiratory system may not be able to deliver all of the prescribed flow rates to the patient or the flow is limited.

It is preferred that the adapter and the main outlet 153 of the valve body 110 are pneumatically sealed such that there is not a significant leak of gas to atmosphere. In some embodiments, if there is a known or expected leak, the flow restriction may be adjusted (for example by altering the size of the tuning orifice) based on this known or expected leak such that expected valve function is maintained.

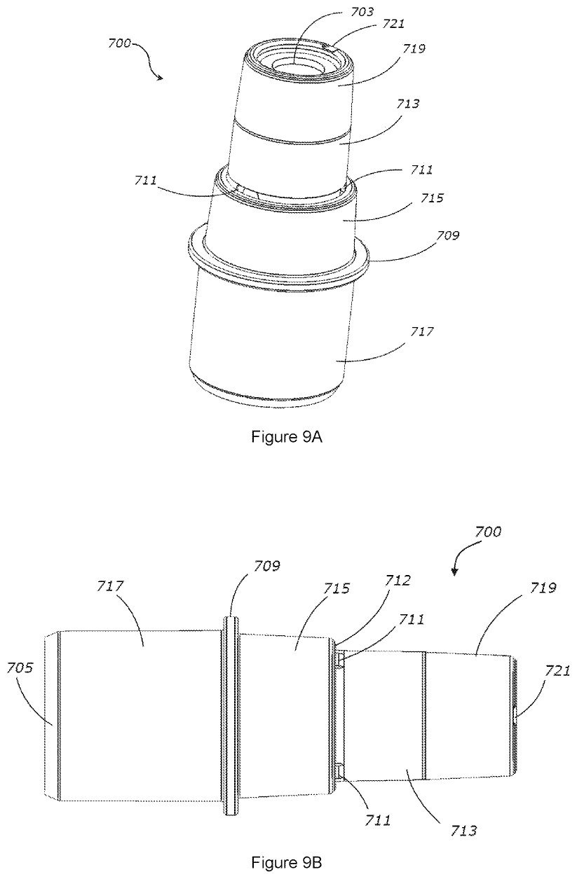

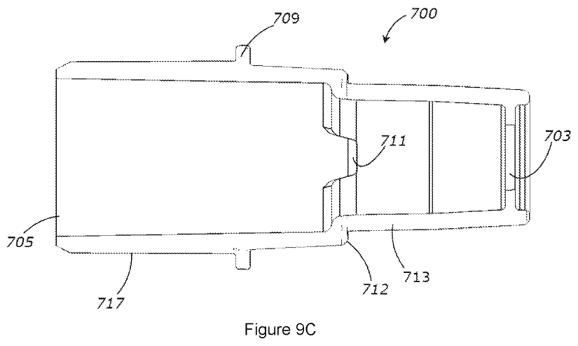

Another embodiment of the adapter will now be described with reference to FIGS. 9A to 9C. The adapter 700 has similar features and functionality to the third embodiment adapter 600, unless described below. Like numbers are used to indicate like parts with the addition of 100.

In the adapter 700 of FIGS. 9A to 9C, the apertures 711 in the wall of the adapter for fluid communication with the FCPRV sensing mechanism 150, are provided in the cavity forming portion of the adapter 713. The apertures 711 are positioned adjacent to the shoulder 712 that is formed between the cavity forming portion 713 and the overlap portion 715. Fluid flow through the apertures 711 is substantially perpendicular to the main flow direction through the adapter 700 from the inlet aperture 703 to the outlet. In some embodiments, the apertures may be provided in the wall of the adapter at, or immediately adjacent and downstream of, the flow restriction.

A moulding notch 721 may be present at the upstream, inlet end of the adapter 700, for ease of manufacturing of the adapter, for example by injection moulding.

In some embodiments described herein, the cavity forming portion may be tapered relative to a direction of gas flow. An example is when the gas flow passage is or comprises a pressure line. The adapter may taper towards a terminal end, from a larger diameter to a smaller diameter.

In some embodiments, the adapter may be configured to be coupled to a pressure relief valve. In particular, the adapter may further comprise an engagement mechanism configured to couple the adapter to a pressure relief valve. Suitable engagement mechanisms include clips, complementary threaded portions, or press fits. In the embodiments shown, the engagement mechanism is a press fit.

In some embodiments, the pressure relief valve may be a flow and/or pressure compensated pressure relief valve. In some embodiments, the pressure relief valve may be a flow compensated pressure relief valve or a pressure compensated pressure relief valve. The pressure line may be in fluid communication with a sensing chamber of the pressure relief valve. The pressure relief valve may comprise a sensing member configured to sense a pressure differential between the sensing chamber and a main gas flow passage that provide gas flow to a patient. Movement of the sensing member changes the venting pressure of a valve member.

In some embodiments, the pressure line is a first pressure line and the adapter further comprise a second pressure line that is upstream of the first pressure line. The first pressure line and the second pressure line may each be coupled to a pressure sensing mechanism.

In some embodiments, the adapter may be configured to be coupled to a respiratory circuit component. For example, the adapter may comprise an engagement mechanism configured to engage the adapter with the respiratory circuit component. Suitable engagement mechanisms include clips, complementary threaded portions, or press fits.

Some of the described embodiments indicate a direction of flow. However, in all described assembly embodiments that the direction of gas flow can be either direction. The terms ‘upstream’ and ‘downstream’ used herein, are dependent on the direction of flow in for example the gas flow passage.

Any one of the adapters described herein may be releasably or permanently secured to, or integral with, the end of a conduit. An example of a conduit 900 is shown in FIG. 6. The adapter may be assembled with the conduit during manufacturing, or after manufacturing. The conduit may be any suitable conduit. The conduit will be chosen or designed depending on a variety of factors. Those factors include the location of the FCPRV in the circuit, and/or the location at which pressure sensing is desired.

The adapter may be configured to releasably attach to the end of an existing conduit to enable the existing conduit to be used with the pressure relief device described herein. The connection between the conduit 900 and the adapter 400 may be by way of an interference fit, for example, where the conduit connecting portion 417 of the adapter is received by the conduit 900 and seals against the internal wall surface of the conduit 900. Alternatively, attachment portion 405 of the adapter 400 may receive the conduit and form an interference fit with the outer surface of the conduit.

This conduit 900 with the adapter 400 is then connected to the FCPRV body, forming a FCPRV and adapter assembly. In a preferred embodiment, the adapter is attached to the end of a conduit during manufacturing. The adapter and the conduit are then connected to the FCPRV by a user. The conduit 900 may be part of a circuit between a flow source and a humidifier or between a pressure relief valve and a humidifier. For example, the conduit may extend from a flow source to a humidifier. The conduit 900 may be referred to as a dry line when it connects an outlet of a flow source or a pressure relief valve to an inlet of a humidifier or humidification chamber, and the gases that it transports is not humidified. Furthermore, additional components may be included to modify the circuit (e.g., a gas flow modulator) and the dry line may extend from a flow source to one of these additional components or from the additional components to a humidifier or humidification chamber. In some embodiments, a gas flow modulator receives a gases flow from a flow source and the adapter and conduit are connected to an outlet of the gas flow modulator to deliver the gases flow from the gas flow modulator to a humidifier or humidification chamber for the gases flow to be humidified. A gas flow modulator may be a gas flow modulator having features described in WO2017/187390, the entirety of which is hereby incorporated by reference herein.

The interaction between the adapter that is integral with or coupled to the dry line and the FCPRV connector is an interference/friction fit in the preferred embodiment. However, other methods may be employed such as a twist/screw attachment or an external engagement mechanism, e.g., adhesives (includes but not limited to glues, chemical bonding, etc), overmoulds and welds.

Each of the adapter described herein allows alterations or modifications to the tuning orifice to be readily made by changing the adapter rather than the entire valve. Further, the adapter described discourage connection of incorrect adapter to the FCPRV connector because the FCPRV will not function as desired unless the adapter is an adapter having the features and functionality of one of the embodiments described here, or unless the adapter is tuned appropriately (e.g. size of the flow restriction) for the resistance to desired flow of the circuit and patient interface.

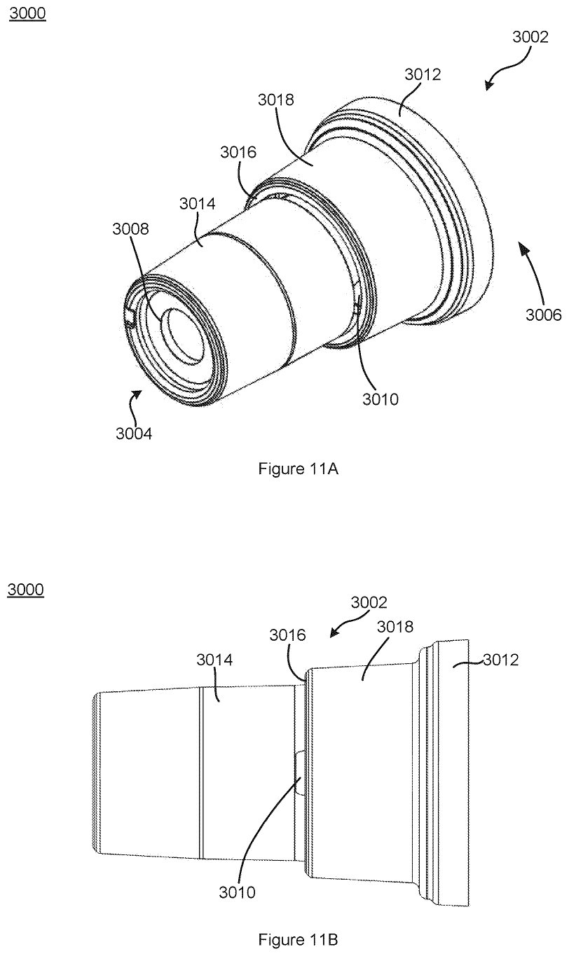

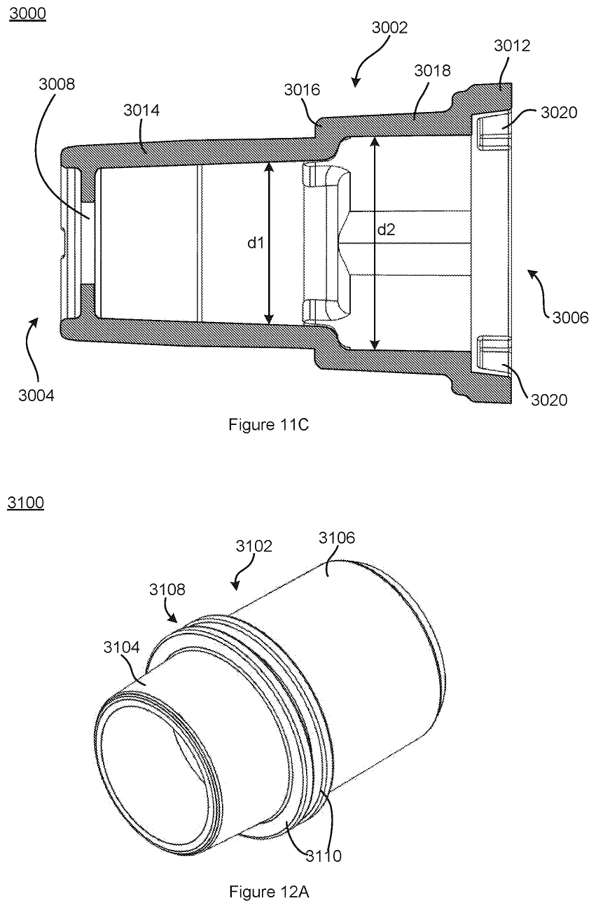

An adapter 3000 for use with a FCPRV 100 according to a further embodiment of the invention will now be described with reference to FIGS. 11A to 11C. The adapter 3000 operates in a similar manner to the adapter embodiments previous described.

As more clearly shown in FIG. 11C, the adapter 3000 includes a hollow body 3002 defining an inlet 3004 and an outlet 3006 configured to provide a gas flow passage therethrough. The inlet 3004 defines an opening 3008. The reduced size of the opening 3008 relative to the cross-sectional dimensions of the hollow body 3002 provides a flow restriction, which restricts gas flow through the gas flow passage. The adapter 3000 also includes an access passage 3010 to the gas flow passage in the form of an aperture in the hollow body 3002. The access passage 3010 is provided downstream of the flow restriction 3008. The adapter 3000 may comprise a plurality of access passages 3010.

The adapter 3000 further includes an attachment portion 3012 proximate the outlet 3006 configured for removable attachment to a rigid adapter base portion 3100 (see FIGS. 12A and 12B).

The hollow body 3002 includes a first portion 3014, a stepped portion 3016, a second portion 3018 and the attachment portion 3012. The first portion 3014 is tapered towards the inlet 3004 to facilitate insertion into a connector associated with the FCPRV 100 (e.g. as described previous with reference to FIGS. 4, 6, 8, and 13 to 19). The first portion 3014 refers to the portion of the adapter 3000 extending from the inlet 3004 to the stepped portion 3016.

As illustrated in FIG. 11C, the internal cross-sectional diameter di of the first portion 3014 immediately upstream of the stepped portion 3016 is smaller than the internal cross-sectional diameter de of the second portion 3018 immediately downstream of the stepped portion 3018. The aperture(s) 3010 for providing the access passage is defined in the stepped portion 3016 (FIGS. 11A and 11B). In other embodiments, one or more apertures for providing access passages may be defined in any one or both of the first portion 3014 or the second portion 3018, in addition to, or alternatively to one or more apertures defined in the stepped portion 3018.

Similarly to the embodiments previously described, during use, the second portion 3018 is adapted for sealing engagement with the FCPRV connector. When the second portion 3018 is sealingly engaged with the FCPRV connector, the first portion 3014 and the stepped portion 3016 forms a cavity with an internal surface of the FCPRV connector such that the cavity is in fluid communication with the gas flow passage via the aperture(s) 3010 (for example, as described with reference to FIGS. 4 and 5).

The attachment portion 3012 includes a shoulder for receiving a rigid base portion 3100. In particular, the attachment portion 3012 may include a plurality of recesses 3020 for engagement with one or more projections 3202 of the rigid base portion 3012 (FIG. 13B) for removable attachment thereto. In one embodiment, the attachment portion 3012 may receive the rigid base portion 3100 (FIGS. 12A and 12B) as described in further detail below.

A rigid base portion 3100 according to one embodiment of the invention is illustrated in FIGS. 12A and 12B. The rigid base portion 3102 includes a hollow body 3102. The hollow body 3102 includes an upstream portion 3104 for removable attachment to the adapter head portion 3000 or 3500 (see FIGS. 16A and 16B), and a downstream portion 3106 for attachment or removable attachment to a flexible conduit. The flexible conduit may be a dry line conduit 900 as previously described and illustrated in FIG. 6.

The rigid base portion 3100 may be an intermediate portion between the adapter head portion 3000 and the flexible conduit 900. In some embodiments, the flexible conduit 900 and the rigid base portion 3100 may be removably attached. In some embodiments, the flexible conduit 900 may be permanently attached to the rigid base portion 3100. In practice, the rigid base portion 3100 and the flexible conduit 900 may be attached together during factory assembly and provided as a single component for use with interchangeable adapter head portions 3000 or 3500. Adapter head portion 3500 is described later with reference to FIGS. 16A and 16B.

The upstream portion 3104 may be partially received within the shoulder of the attachment portion 3012 of the adapter 3000 (also referred to herein as an adapter head portion 3000). In some embodiments, the attachment between the adapter head portion 3000 and the rigid base portion 3102 is a sealing engagement by friction fit. In other embodiments, the adapter head portion 3000 may be attached to the rigid base portion 3100 by any suitable means, for example, clips, complementary threaded portions, press fits and the like, or any combination thereof.

In another embodiment, the upstream portion 3104 may be partially received within the attachment portion 3524 of the adapter 3500 (also referred to herein as an adapter head portion 3500). The attachment between the adapter head portion 3500 and the rigid base portion 3102 may be a sealing engagement by friction fit. In other embodiments, the adapter head portion 3500 may be attached to the rigid base portion 3100 by any suitable means, for example, clips, complementary threaded portions, press fits and the like, or any combination thereof.

The rigid base portion 3100 further includes a channel 3108 for receiving a tether, so that the adapter head portion 3000 or 3500 can be tethered to the rigid base portion 3100, for example as shown in FIGS. 13A and 13B. The channel 3108 is formed by a pair of ridges 3110 extending outwardly from the hollow body 3102 of the rigid base portion 3100. As described below with reference to FIGS. 16A and 16B, the adapter head portion 3500 may also include a corresponding channel 3520 for receiving the tether.

Advantageously, the adapter head portions 3000, 3500 and the rigid base portion 3100 provides a versatile adapter assembly, whereby a plurality of different adapter head portions 3000, 3500 providing different levels of flow restriction can be interchangeably used with the same rigid base portion 3100 to satisfy different requirements of the respiratory system 10. For example, different adapter head portions 3000, 3500 can be provided with different levels of flow restriction, each flow restriction being tuned to deliver optimal performance when used with different respiratory system configurations, which have different system pressures (also referred to herein as resistance to flow RTF) downstream of the FCPRV. The different system pressures downstream of the FCPRV (referred to herein as ‘downstream RTF’) may be due to different component shapes and sizes, for example, conduits of different cross-sectional shape/dimension and/or length, and different types of patient interface (e.g., an adult patient interface, or a paediatric patient interface).

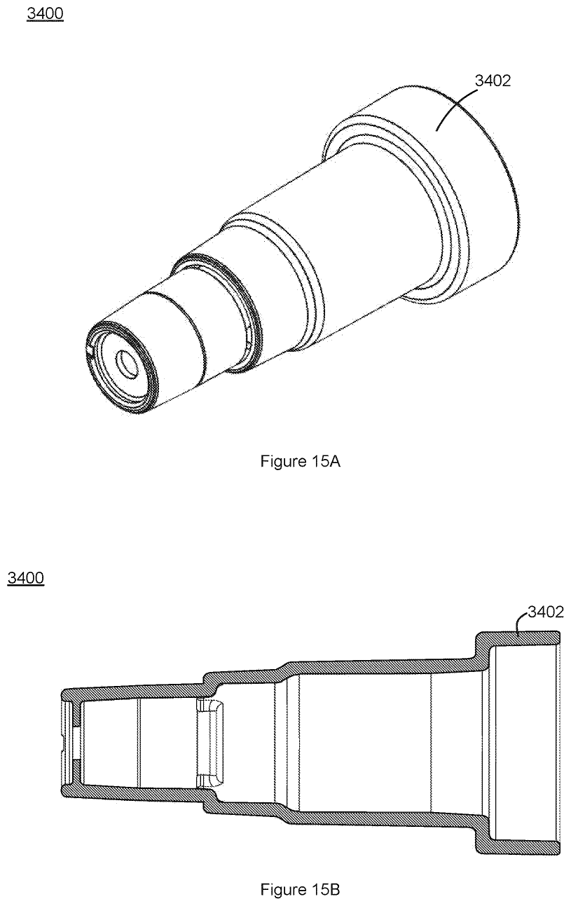

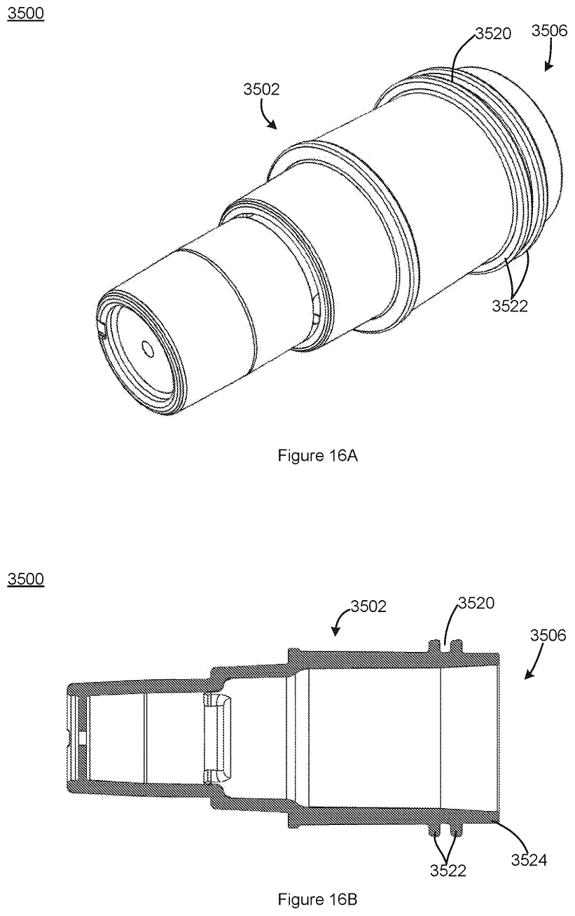

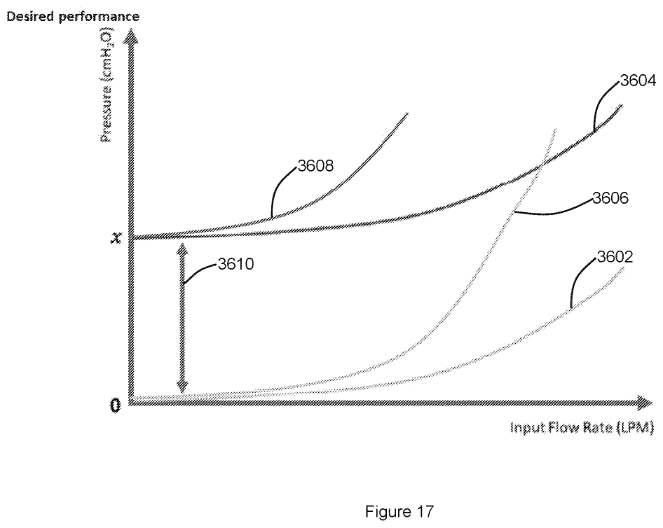

In one example use case scenario, the pressure and flow performance requirement of the respiratory system 10 for an adult patient may be different to that for a paediatric patient. In particular, a paediatric patient interface typically includes smaller diameter flow paths when compared with an adult patient interface. As such, a respiratory system 10 using a paediatric patient interface would typically have a higher resistance to flow downstream of the FCPRV 100, which can result in a reduction of the maximum deliverable flow rate of the respiratory system 10 if the paediatric patient interface is used with an adapter tuned for a system having an adult patient interface. As previously explained with reference to FIG. 8, the flow and pressure characteristics of the system relates to the gradient of the FCPRV relief pressure response curve, and the gradient can be adjusted by adjusting the size of the inlet opening 3008 on the adapter head portion 3000. The gradient represents the rate of change of the FCPRV relief pressure with increasing flow rate. A higher gradient typically indicates a greater rate of increase in the FCPRV relief pressure with increasing flow rate. Accordingly, by providing different adapter head portions 3000 (or adapter head portion 3500) with different sized inlet openings 3008 for removable attachment to the rigid base portion 3102, the respiratory system 10 can be tuned to provide optimal performance for a wide range of different respiratory systems having different downstream RTF, for example in part due to using different patient interface types, ranging from those configured for different patient populations, such as neonatal and paediatric patients to adult patients. Generally, an adapter head portion 3000 tuned for a system having higher downstream RTF (such as a system using a paediatric patient interface) provides a smaller inlet opening 3008 compared with an adapter head portion 3000 tuned for a system having lower downstream RTF (such as a system using an adult patient interface). A comparison of the FCPRV and adapter assembly performance with changing inlet opening 3008 size will be discussed in further detail with reference to FIG. 17.

A further embodiment of an adapter assembly 3200 is illustrated in FIGS. 13A and 13B, in which the adapter head portion 3000 is tethered to the rigid base portion 3204 via a tether 3206. The tether 3206 may be integral or separately mounted to the head portion 3000 and rigid base portion 3204. Features of the adapter head portion 3000 are similar to those previously described with reference to FIGS. 11A to 11C.

The rigid base portion 3204 may include projections 3202 for insertion into corresponding recesses 3020 of the head portion 3000 to facilitate removable mounting of the head portion 3000 to the base portion 3204. As previously mentioned, the head and base portions 3000, 3204 can be removably attached by any suitable means, including interference fit, snap fit, via complimentary threaded portions, or any combination thereof.

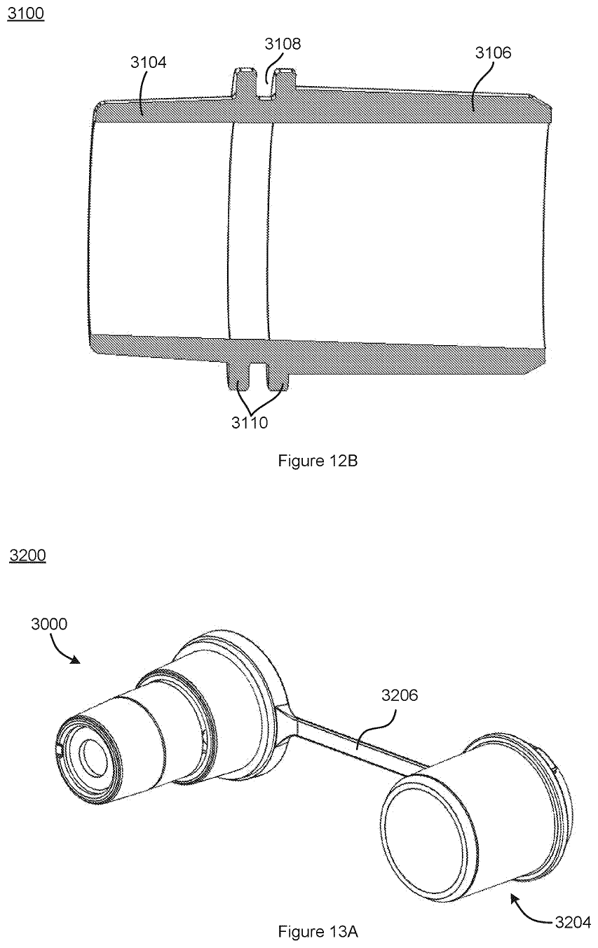

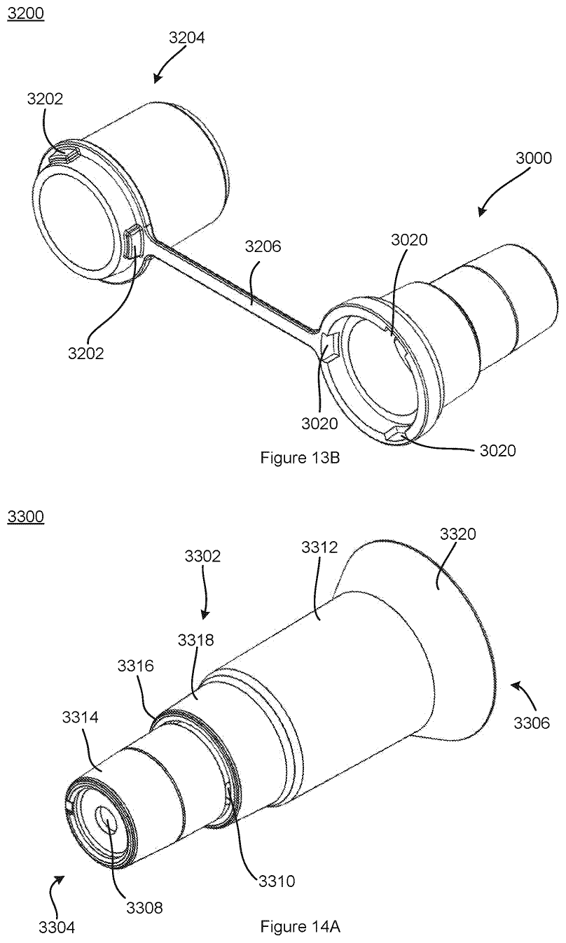

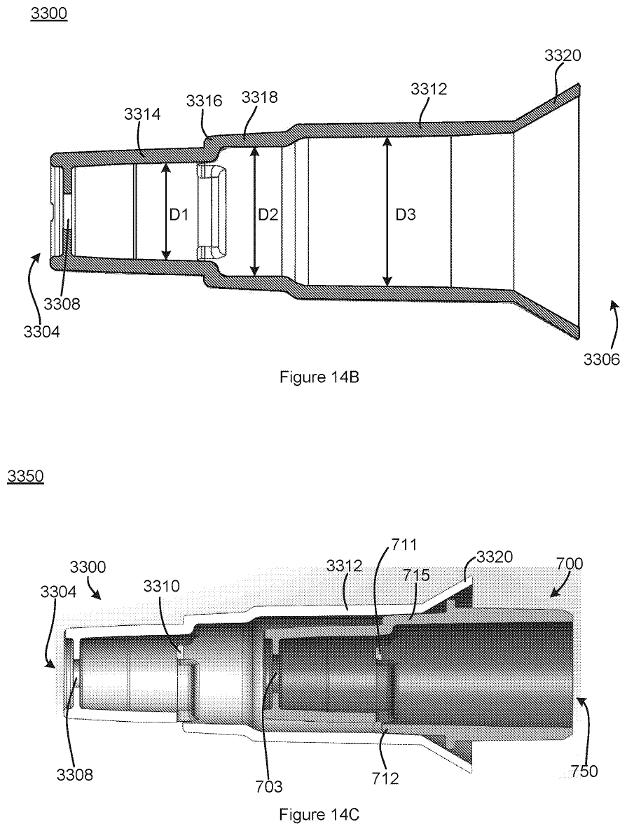

An adapter 3300 for use with a FCPRV 100 according to a further embodiment of the invention will now be described with reference to FIGS. 14A to 14B. The adapter 3300 operates in a similar manner to the adapter embodiments previous described.

The adapter 3300 includes a hollow body 3302 defining an inlet 3304 and an outlet 3306 configured to provide a gas flow passage therethrough. The inlet 3304 defines an opening 3308. The reduced size of the opening 3308 relative to the cross-sectional dimensions of the hollow body 3302 provides the flow restriction, which restricts gas flow through the gas flow passage. The adapter 3300 also includes an access passage 3310 to the gas flow passage in the form of an aperture in the hollow body 3302. The access passage 3310 is provided downstream of the flow restriction 3308. The adapter 3300 may comprise a plurality of access passages 3310.

The adapter 3300 further includes a mounting portion 3312 proximate the outlet 3306 configured for removable mounting to a second like adapter (such as adapter 700 shown in FIG. 14C). The second like adapter may be any one of the adapters previously described herein.

The hollow body 3302 includes a first portion 3314, a stepped portion 3316, a second portion 3318, and the mounting portion 3312. The first portion 3314 is tapered towards the inlet 3304 to facilitate insertion into a connector associated with the FCPRV 100.

As illustrated in FIG. 14B, the internal cross-sectional diameter D₁ of the first portion 3314 immediately upstream of the stepped portion 3316 is smaller than the internal cross-sectional diameter D₂ of the second portion 3318 immediately downstream of the stepped portion 3316. The aperture(s) 3310 for providing the access passage is defined in the stepped portion 3316 (FIG. 14A). As mentioned, one or more apertures may additionally, or alternatively, be provided in other parts of the hollow body 3302, for example in the first portion 3314 or second portion 3318.

Similarly to the embodiments previously described, during use, the second portion 3318 is adapted for sealing engagement with the FCPRV connector. When the second portion 3318 is sealingly engaged with the FCPRV connector, the first portion 3314 and the stepped portion 3316 forms a cavity with the FCPRV connector such that the cavity is in fluid communication with the gas flow passage via the aperture(s) 3310 (for example, as described with reference to FIGS. 4 and 5).

The mounting portion 3312 is further downstream of the second portion 3318 and has an internal cross-sectional diameter D₃ greater than that of the first portion 3314 and the second portion 3318, for receiving the second like adapter 700.

As more clearly shown in FIG. 14C, the mounting portion 3312 is configured for sealing engagement with a corresponding hollow body of the second like adapter 700 proximate a corresponding stepped portion 712 of the second like adapter 700. In particular, the inlet 703 and access passage aperture(s) 711 of the second like adapter 700 are fully encapsulated in the hollow body 3302 of the adapter 3300 when the adapter 3300 is mounted on the second like adapter 700.

In one embodiment, the second like adapter 700 has an inlet opening 703 tuned for an adult user interface. The inlet opening 3308 of adapter 3300 is smaller than the inlet opening 703 of the second like adapter 700. Accordingly, the flow restriction provided by adapter 3300 is greater than the flow restriction provided by the second like adapter 700. When adapter 3300 is mounted over the second like adapter 700 as illustrated in FIG. 14C, the adapter assembly 3350 comprising two adapters 3300, 700 function as a single adapter unit that is tuned for a respiratory system having a higher downstream RTF (i.e. a system using paediatric user interface). In use, the inlet 3304 of the adapter 3300 is the inlet of the adapter assembly 3350, and the outlet 750 of the second like adapter 700 is the outlet of the adapter assembly 3350. For the adapter assembly 3350, gas flow enters the assembly 3350 via inlet 3304, and flows through the gas flow passage via inlet 703 and apertures 711 of the second like adapter 700 before reaching the outlet 750. The gradient of the FCPRV relief pressure response curve can be determined by the size of the inlet opening 3308 of adapter 3300 when the adapter assembly 3350 is used. In this manner, the pressure and flow performance parameters of a respiratory system tuned to function with a system having a particular downstream RTF by using adapter 750 with the FCPRV 100 can be easily and conveniently modified to function with a system having a different downstream RTF (e.g. when an adult user interface is exchanged with a paediatric user interface in the respiratory system) by mounting adapter 3300 over the adapter 750 as shown in FIG. 14C.

In the particular embodiment shown in FIG. 14C, the adapter 3300 includes a flared portion 3320 proximate the outlet 3306 of the hollow body 3302 to facilitate receiving the second like adapter 700 therein.

In another embodiment as illustrated in FIGS. 15A and 15B, the adapter 3400 includes a stepped flange 3402 proximate the outlet 3306 of the hollow body 3302 to facilitate receiving the second like adapter therein. Like components of the adapter 3400 function in the same manner as those previously described with reference to adapter 3300 in FIGS. 14A to 14C.

In another embodiment as illustrated in FIGS. 16A and 16B, the adapter 3500 includes a channel 3520 proximate the outlet 3506 of the hollow body 3502 for receiving a tether (not shown). The channel 3520 is defined between two parallel ridges 3522 projecting outwardly from the hollow body 3502. Like components of the adapter 3500 function in the same manner as those previously described with reference to adapter 3300 in FIGS. 14A to 14C. The tether may enable the adapter 3500 to be tethered to a flexible conduit (e.g. dry line), or a second like adapter, so that the adapter 3500 is readily available and can be mounted to the second like adapter to modify the pressure and flow performance parameters of a respiratory gas delivery system 10 when required.

In an alternative configuration, the adapter 3500 may be an adapter head portion for removable mounting to a rigid base portion 3100 (FIGS. 12A and 12B). In this configuration, the adapter 3500 may be tethered to the rigid base portion 3100. The outlet 3506 of adapter 3500 may include attachment portion 3524 for removable attachment to the rigid base portion 3100. In particular, the upstream portion 3104 of the rigid base portion 3100 can be at least partially received in the attachment portion 3524 by friction fit, such that the attachment portion 3524 of the adapter head 3500 forms sealing engagement with the upstream portion 3104 of the rigid base portion 3100.

The system and relief pressure vs flow rate response curves 3600 for adult and paediatric patients as modified using the different adapter and adapter assemblies described herein will now be discussed with reference to FIG. 17.

The system pressure downstream of a FCPRV 100 (also referred to as the system resistance to flow or RTF) with respect to increasing input flow rate when the system 10 is used with an adult user interface is represented using curve 3602. The relief pressure of the FCPRV 100 with respect to increasing input flow rate when the system 10 is used with the adult user interface is represented using curve 3604. As previously discussed, the FCPRV 100 and adapter assembly is capable of delivering variable relief pressure 3604 with varying flow rate so that gas can be delivered to the patient at a range of different flow rates, whilst maintaining a maximum deliverable pressure to the patient 3610. The maximum deliverable pressure 3610 for any given flow rate being the difference between the FCPRV 100 relief pressure 3604 and the downstream system pressure 3602 at the given flow rate.

As indicated by curve 3606, the downstream RTF of the system 10 when used with a paediatric interface may increase more rapidly with increasing flow rate, as compared with curve 3602. Accordingly, it would be desirable to increase the gradient of the FCPRV relief pressure response curve for a system 10 having higher downstream RTF (such as a system with a paediatric interface).

By increasing the flow restriction in the adapter, for example by reducing the size of the inlet opening of the adapter (for example, see inlet opening 3308 as shown in FIGS. 14A to 14C), a pressure drop created across the flow restriction increases in magnitude, and gradient of the FCPRV relief pressure response curve also increases. When the FCPRV 100 is used together with an adapter providing a smaller inlet opening, the relief pressure of the FCPRV 100 with respect to increasing input flow rate is represented using curve 3608. The size of the inlet opening can therefore be tuned to ensure that a maximum deliverable patient pressure 3610 (being the pressure difference between curves 3608 and 3606 for a given flow rate) does not exceed a predetermined value for all flow rates provided by the system 10 (e.g. 20 cmH₂O), whilst enabling the system 10 to deliver a wide range of flow rates to the paediatric patient via a paediatric patient interface.

Whilst FIG. 17 is described with reference to a specific example where the downstream RTF is changed by changing the patient interface from an adult patient interface to a paediatric patient interface, it is understood that the downstream RTF can be affected or changed by a variety of different factors, including patient interface type, the number and type of downstream components and modules, as well as the size, shape, and length of flow paths provided by the downstream components and modules.

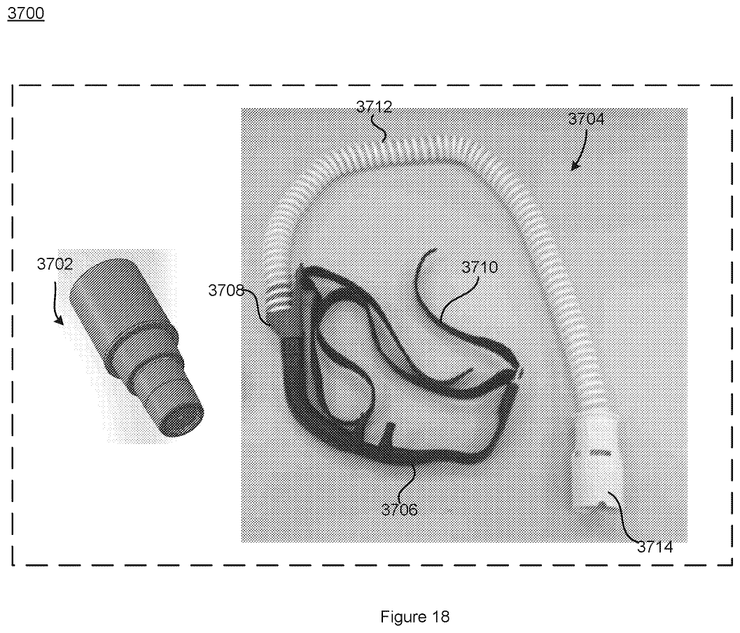

FIG. 18 illustrates a kit 3700 for a respiratory gas delivery system 10 for delivering a gas flow to a patient 16. The kit includes a flow modifying adapter 3702 such as an adapter or adapter assembly as herein described configured for removable connection to a flow modulator. The flow modulator may include the flow source 12 and/or FCPRV 100. As previously mentioned, the system 10 typically includes a combination of single-use and multi-use modules and components. The kit 3700 may be provided as single-use components for a particular patient. The flow modifying adapter may be a single use or a multi-use component. The single-use components may interface with multi-use components/modules in the system 10. To indicate that the flow modifying adapter 3702 is tuned for use with the patient interface 3704, the flow modifying adapter 3702 and the patient interface 3704 includes matching visual indicators.

In one embodiment, the flow modifying adapter 3702 and a connector 3708 of the patient interface 3704 may be moulded using the same-coloured plastic such that at least parts of the two components are the same colour. Alternatively, or in addition, the nasal cannula 3706 may be the same colour as the flow modifying adapter 3702.

Alternatively, or in addition, a conduit 3712 associated with the patient interface 3704 and/or the connector 3714 associated with the conduit 3712 may be the same colour as the flow modifying adapter 3702. In some embodiments, matching labelling such as ‘ADULT’, ‘PAEDIATRIC’, ‘XS’, ‘S’, ‘M’, ‘L’, or any other suitable visual indicia may be used alternatively or in addition to the colour indicators.

Advantageously, a flow modifying adapter tuned to operate in a respiratory system with a particular type of patient interface can be provided with matching visual indicators (e.g., colour indicators) to assist a clinician in identifying the correct adapter for use with a specific patient interface. In practice, a first kit comprising a first flow modifying adapter and a first patient interface in which the flow modifying adapter is tuned to operate with the first patient interface may be provided with matching colour indicators in a first colour; and a second kit comprising a second flow modifying adapter and a second patient interface in which the second flow modifying adapter is tuned to operate with the second patient interface may be provided with matching colour indicators in a second colour, which is different to the first colour.

Whilst not shown in FIG. 18, the kit 3700 can include any one or more of the following additional components:
a filter for the patient interface.
a flexible conduit for connection with the flow modifying adapter,
a humidification chamber for a humidifier, and
an inspiration tube for connection with the patient interface.

The filter may be adapted for placement in a connector 3714 of the patient interface 3704 such that the filter is placed between the patient interface 3704 and the inspiration tube.

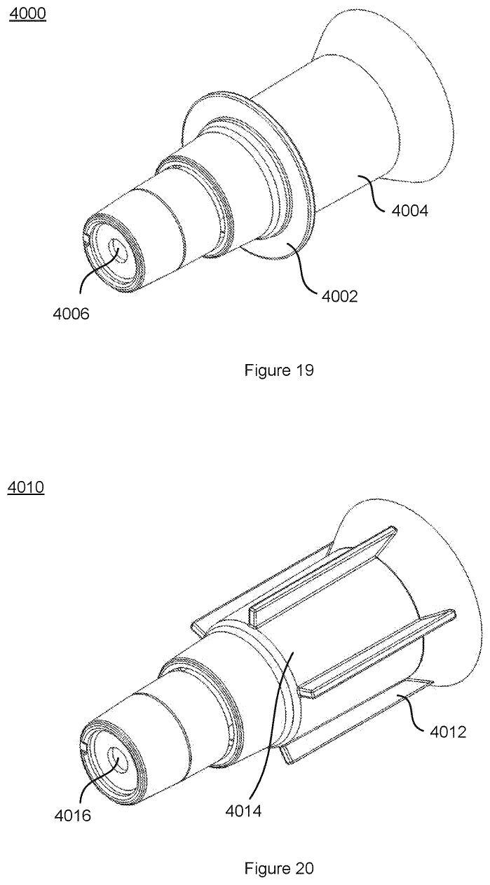

FIG. 19 illustrates a further embodiment of an adapter 4000. The adapter 4000 further includes a radial projection 4002 extending radially outwardly from a hollow body 4004. Like components of the adapter 4000 function in the same manner as those previously described with reference to adapter 3300 in FIGS. 14A to 14C. The radial projection 4002 advantageously acts as a safety feature and serves to prevent incorrect attachment of another like adapter (e.g. adapter 700 or 3300 as shown in FIG. 14C) over the adapter 4000. In this embodiment, the size of the inlet opening 4006 may be smaller than that of the like adapter 700 (which is tuned for an adult user interface) such that when the adapter 4000 is mounted over the like adapter 700 the adapter assembly comprising both adaptors 4000, 700 function as a single adapter unit that is tuned for a respiratory system having a higher downstream RTF (i.e. a system using a paediatric user interface) in a similar manner to the adapter assembly 3350 described with reference to FIG. 14C. In other embodiments, the adapter 4000 may include a plurality of radial projections 4002 spaced along the hollow body 4004.

FIG. 20 illustrates yet another embodiment of an adapter 4010. The adapter 4010 includes a plurality of longitudinal extensions (ribs) 4012 extending generally along a lengthwise direction of the hollow body 4014. In other embodiments, the ribs may extend diagonally or helically across the body 4014 of the adapter 4010. Like components of the adapter 4010 function in the same manner as those previously described with reference to adapter 3300 in FIGS. 14A to 14C. The longitudinal extensions 4012 function in a similar way to the radial projection 4002 of the adapter 4000 in FIG. 19, and acts as a safety feature to prevent incorrect attachment of another like adapter (e.g. adapter 700 or 3300 as shown in FIG. 14C) over the adapter 4000. Similarly to adapter 4000, the size of the inlet opening 4016 is configured to be smaller than that of the like adapter 700 (which is tuned for an user interface) such that when the adapter 4010 is mounted over the like adapter 700 the adapter assembly comprising both adaptors 4010, 700 function as a single adapter unit that is tuned for a respiratory system having a higher downstream RTF (i.e. a system using a paediatric user interface) in a similar manner to that described with reference to FIG. 14C. In other embodiments, it will be appreciated that the adapter 4010 may include a single longitudinal extension 4012.

As previously mentioned, the flow restriction provided by each adapter 4000, 4010 or each adapter assembly 3350 as illustrated is typically tuned so as to provide a specific and desired operation for a respiratory system 10. It is desirable to prevent attachment of another like adapter (e.g. 700 or 3300) over an adapter specifically tuned to operate either independently or as an external adapter in an assembly as shown in FIG. 14C. Incorrect attachment of an adapter 700, 3300 over another adapter 4000, 4010 that is tuned to be an externally mounted adapter in an adapter assembly would result in undesirable changes to RTF downstream of the adapter assembly. This is because an incorrectly stacked adapter assembly would provide an RTF that is incompatible with the respiratory system configuration.

This incorrect RTF could prevent the associated pressure valve 100 from operating correctly, for example, causing the pressure valve 100 to vent at an incorrect pressure. This could result in the system underdelivering flow to a patient during therapy or over pressurising the patient. Providing the incorrect RTF could also result in overpressure potentially causing damage to other components in the respiratory system, such as the pressure valve 100 or the flow source 12.

In some embodiments, the adapter may further include a flow restriction adjustment mechanism to enable flexible adjustment of the flow restriction provided by the adaptor. This may be achieved by changing the resistance to flow provided by the adapter, for example by adjusting the size and/or configuration of the inlet opening. Typically, the size of the inlet opening can be adjusted by adjusting the cross-sectional area of the inlet opening.

The flow restriction of an adapter may be adjusted manually by a user so as to provide the required FCPRV relief pressure response characteristics for a particular patient interface 15 (e.g. adult or paediatric patient interface). As previously discussed with reference to FIGS. 8 and 17, adjustment of the flow restriction of the adapter allows convenient tuning of the RTF downstream of the pressure valve so as to alter the gradient of the relief pressure response curve (FIG. 17) of the pressure valve so that a desired relief pressure vs flow response can be achieved to suit a particular patient undergoing specific treatment. In particular, it would be desirable to enable flexible adjustment of the flow restriction for an adapter so that a clinician can conveniently tune the relief pressure response each time that a new patient interface is used to ensure that a maximum deliverable patient pressure 3610 (being the pressure difference between curves 3608 and 3606 for a given flow rate) does not exceed a predetermined value for all flow rates provided by the system 10 (e.g. 20 cmH₂O).

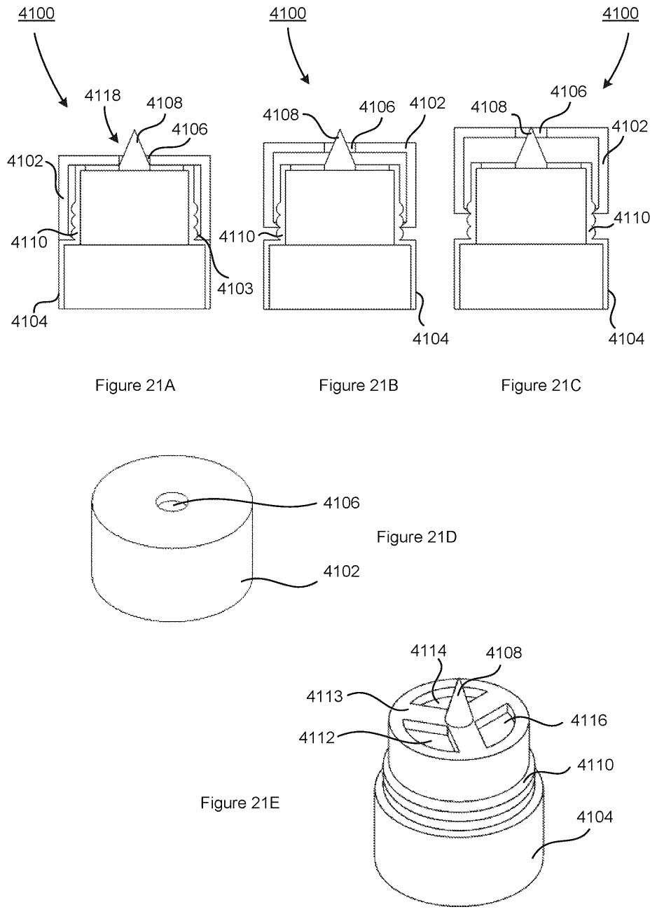

Some examples of different flow restriction adjustment mechanisms will be described below with reference to FIGS. 21A to 34C.

As illustrated in FIGS. 21A to 21E, the flow restriction adjustment mechanism 4100 includes a terminal portion 4102 removably and/or moveably attached to an adapter end portion 4104. The adapter end portion 4104 may be integral with, or separately provided for attachment to, the hollow body (e.g. 4004) of an adapter in accordance with any one of the adapters described herein. The adapter end portion 4104 may be upstream of, or may comprise, the access passage(s).

The terminal portion 4102 is in the form of a cap configured for engagement via a ridged portion 4110 provided by the adapter end portion 4104. As shown in FIGS. 21A to 21C, the cap 4102 includes a lip 4103 configured to engage with recesses between adjacent ridges in the ridged portion 4110. Translation of the lip 4102 across the ridged portion 4110 enables the lip 4103 to move between different ridges in the ridged portion 4110, thereby allowing discrete adjustment of the cap 4102 position relative to the adapter end portion 4104. In some embodiments, more than one cap 4102 may be provided with varying sized openings 4106 for use with the adapter end portion 4104, to provide a greater the adjustment range for the flow restriction adjustment mechanism 4100.

In other embodiments, the cap 4102 may include a complementary threaded portion for threading engagement with the adapter end portion 4104. In some embodiments, the cap 4102 may be moveably mounted to the adapter end portion 4104 via other means, for example, via press fit or the like.

The cap 4102 defines an opening 4106, and the adapter end portion 4104 provides a protrusion 4108. In the particular embodiment shown, the protrusion 4108 is tapered. More specifically, the protrusion 4108 has a conical shape. However, it will be appreciated that the protrusion 4108 can have any suitable shape, for example the protrusion 4108 may be cylindrical as shown in FIG. 23B. Moreover, a tapered protrusion 4108 may provide for a wider range of adjustment than a non-tapered protrusion, which may be more desirable in some applications. The opening 4106 is configured for alignment with the protrusion 4108 and vice versa when the cap 4102 is mounted to the adapter end portion 4104 as more clearly shown in FIGS. 21A to 21C. As described in further detail below, movement of the cap 4102 relative to the adapter end portion 4104 provides an adjustable inlet 4118 to the adapter associated with the flow restriction adjustment mechanism 4100. As more clearly shown in FIG. 21E, the adapter end portion 4104 defines a plurality of apertures 4112, 4114, 4116 to facilitate gas flow via the adjustable inlet 4118. Whilst three apertures 4112, 4114, 4116 are shown in FIG. 21E, any suitable number of apertures may be provided by the adapter end portion 4104. For example, the adapter end portion 4104 may provide one or more apertures. The apertures 4112, 4114, 4116 are formed by the protrusion 4108 suspended by arms 4113. Whilst three arms 4113 are shown in FIG. 21E, it is to be understood that any number of arms 4113 may be provided. For example, one or more arms 4113 may be provided.

Typically, the total cross-sectional area of the apertures 4112, 4114, 4116 may be equal to or greater than the cross-sectional area of the opening 4106 so that adjustment of the flow restriction is predominantly defined by the relative positions between the opening 4106 and the protrusion 4108.

As illustrated in the cross-sectional views of the flow restriction adjustment mechanism 4100 in FIGS. 21A to 21C, movement of the cap 4102 towards (FIG. 21A) and away (FIG. 21C) from the adapter end portion 4104 provides adjustment to the overall size and configuration of the adjustable inlet 4118. In particular, when the protrusion 4108 is fully inserted in the opening 4106 as shown in FIG. 21A (minimum distance between the cap 4102 and the adapter end portion 4104), maximum flow restriction is provided through the adjustable inlet 4118. In some embodiments, it may be desirable to prevent sealing between the cap 4102 and the adapter end portion 4104 to avoid creating an occlusion in the respiratory system, which could cause safety issues. Accordingly, a minimum level of gas flow may be permitted through the inlet 4118 in the position shown in FIG. 21A. When the opening 4106 is spaced from the protrusion 4108 at a maximum distance (FIG. 21C), a minimum flow restriction is provided by the adjustable inlet 4118. In the condition shown in FIG. 21C, gas flow enters the adapter via opening 4106 of the cap 4102 and apertures 4112, 4114, 4116 of the adapter end portion 4104. A medium level of flow restriction may be provided by the adjustable inlet 4118 when the cap 4102 is at an intermediate position with respect to the adapter end portion 4104 (FIG. 21B) between the end positions shown in FIGS. 21A and 21C. In this manner, the flow restriction adjustment mechanism 4100 may provide discrete adjustment of the flow restriction by enabling adjustment of the inlet 4118 when the cap 4102 is moved relative to the adapter end portion 4104. Whilst three discrete positions are illustrated in FIGS. 21A to 21C, it is to be understood that any suitable number of discrete position adjustments can be provided by providing two or more ridges in the ridged portion 4110, In other embodiments, continuous adjustment may be provided, for example if the cap 4102 is configured for threading engagement with the adapter end portion 4104.

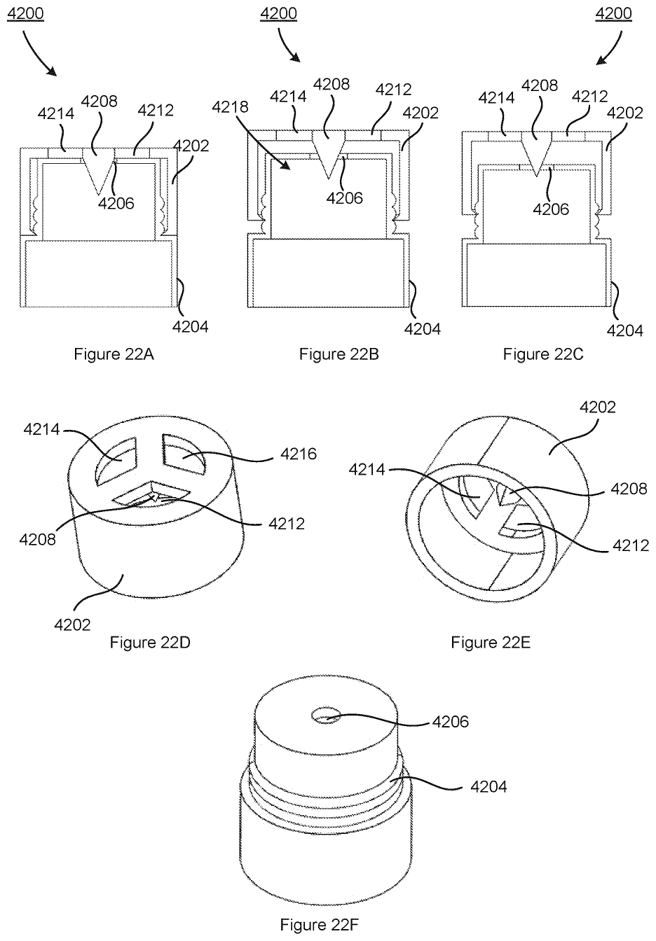

In an alternative embodiment as illustrated in FIGS. 22A to 22F, the flow restriction adjustment mechanism 4200 works in a similar manner to the flow restriction adjustment mechanism 4100 previously described with reference to FIGS. 21A to 21E. Accordingly, like features in Figures shown in FIGS. 22A to 22E function in a similar manner to those described above with reference to FIGS. 21A to 21E.

In the flow restriction adjustment mechanism 4200 the projection 4208 and apertures 4212, 4214, 4216 are provided on the cap 4202 instead of the adapter end portion 4204, and the opening 4206 is provided on the adapter end portion 4204 instead of the cap 4202. However, the same concept of operation applies to both embodiments of the restriction adjustment mechanism 4000, 4200.

As illustrated in the cross-sectional views of the flow restriction adjustment mechanism 4200 in FIGS. 22A to 22C, the projection 4208 is configured for alignment with the opening 4206 and vice versa. Movement of the cap 4202 towards and away from the adapter end portion 4204 provides continuous and/or discrete adjustment to the overall size and configuration of the adjustable inlet 4218.

In a further variation of the restriction adjustment mechanism 4300 as shown in FIGS. 23A and 23B, the protrusion 4308 provided by the adapter end portion 4304 may be of any suitable shape and configuration. In the embodiment shown in FIG. 23B, the protrusion 4308 is generally cylindrical. In other embodiments, the protrusion may have a rectangular, triangular, or any shaped cross section. A skilled person would appreciate that the operation of the restriction adjustment mechanism 4300 follows the same concept as that described with reference to FIGS. 21A to 21E. Similarly, the protrusion 4308 may be provided by the cap 4302, in a similar manner to FIGS. 22A to 22F.

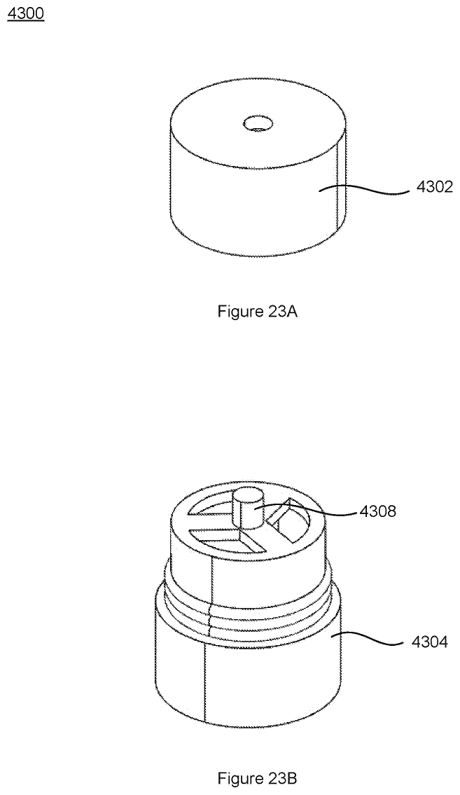

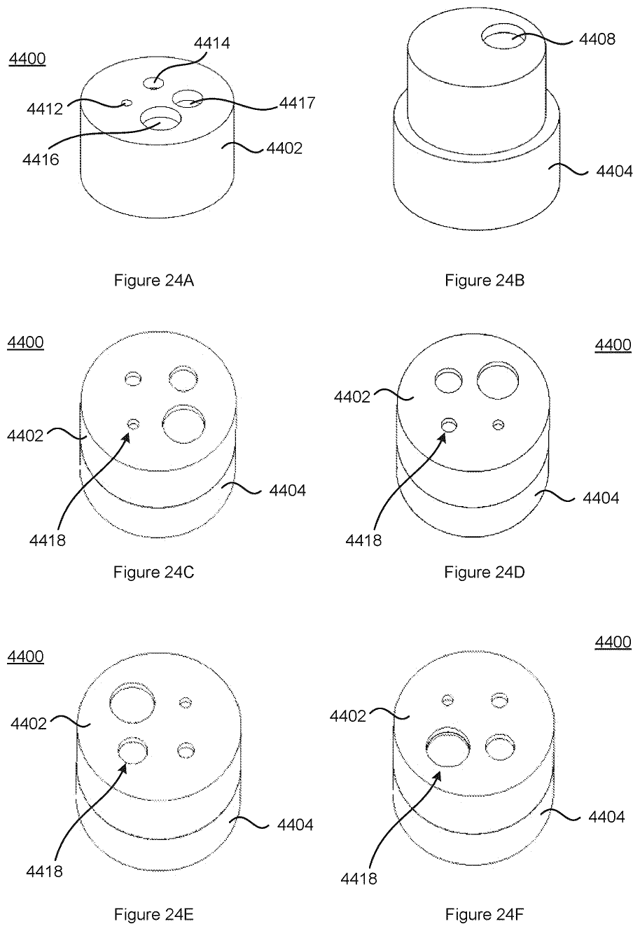

As illustrated in FIGS. 24A to 24F, the flow restriction adjustment mechanism 4400 according to another embodiment includes a terminal portion 4402 removably and/or moveably attached to an adapter end portion 4404. The adapter end portion 4404 may be integral with, or separately provided for attachment to, the hollow body (e.g. 4004) of an adapter in accordance with any one of the adapters described herein. The adapter end portion 4404 may be upstream of, or may comprise, the access passage(s).

The terminal portion 4402 is in the form of a cap configured for moveable engagement relative to the adapter end portion 4404. It will be appreciated that any suitable engagement mechanism may be used. For example,, press fit engagement, clipping engagement via ridges and/or channels and the like. Typically, the cap 4402 is mounted on the adapter end portion 4404 such that the cap 4402 is rotatable relative to the adapter end portion 4404. A lateral distance between the cap 4402 and the adapter end portion 4404 may be fixed.

The cap 4402 defines a plurality of openings 4412, 4414, 4416, 4417 varying in size, and the adapter end portion 4404 provides an aperture 4408. As more clearly shown in FIGS. 24C to 24F, rotation of the cap 4402 relative to the adapter end portion 4404 provides alignment between each of the plurality of openings 4412, 4414, 4416, 4417 and the aperture 4408 to provide a variable sized inlet 4418 for the associated adapter. Whilst four openings 4412, 4414, 4416, 4417 are illustrated in FIG. 24A, it will be understood that any suitable number of openings may be provided to provide the range of different sizes for the inlet 4418 as required. In some embodiments, misalignment between the aperture 4408 and openings 4412, 4414, 4416, 4417 may provide additional levels of adjustment. Typically, friction between the cap 4402 and the adapter end portion 4404 is sufficient to avoid inadvertent movement between the cap 4402 and the adapter end portion 4404 in use.

Typically, the aperture 4408 is larger or similar in size than any one of the openings 4412, 4414, 4416, 4417 such that when each one of the openings 4412, 4414, 4416, 4417 is generally aligned with the aperture 4408 as shown in FIGS. 24C to 24F, the effective size of the inlet is determined by the size of the respective aligned opening 4412, 4414, 4416, 4417.

In an alternative embodiment as shown in FIGS. 25A and 25B, the flow restriction adjustment mechanism 4500 functions in a similar manner to the flow restriction adjustment mechanism 4400 as described above with reference to FIGS. 24A to 24F. In the flow restriction adjustment mechanism 4500, the plurality of openings 4512, 4514, 4516, 4517 are provided by the adapter end portion 4504 rather than the cap 4502, and the aperture 4508 is provided on the cap 4502 instead of the adapter end portion 4504. However, the concept of operation for the flow restriction adjustment mechanism 4500 is the same as that described in relation to the flow restriction adjustment mechanism 4400. In particular, aperture 4508 is typically the same size or larger than the largest opening from the plurality of openings 4512, 4514, 4516, 4517. Rotation of the cap 4502 relative to the adapter end portion 4504 allows the aperture 4508 to be generally aligned with any one of the openings 4512, 4514, 4516, 4517 as required. The size of the effective inlet associated with the adapter provided by the flow restriction adjust mechanism 4500 can therefore be defined by the size of the respective aligned opening 4512, 4514, 4516, 4517. Other like features of the flow restriction adjustment mechanism 4500 function in a similar manner to those described previously with reference to FIGS. 24A to 24F.

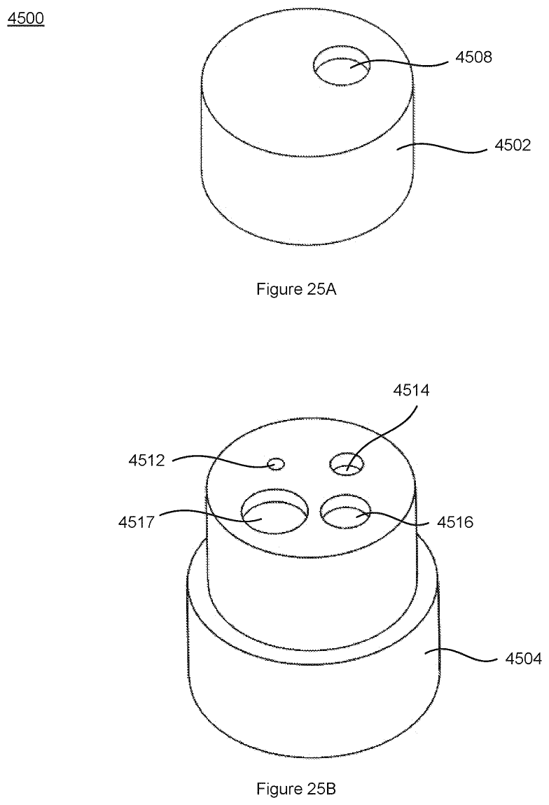

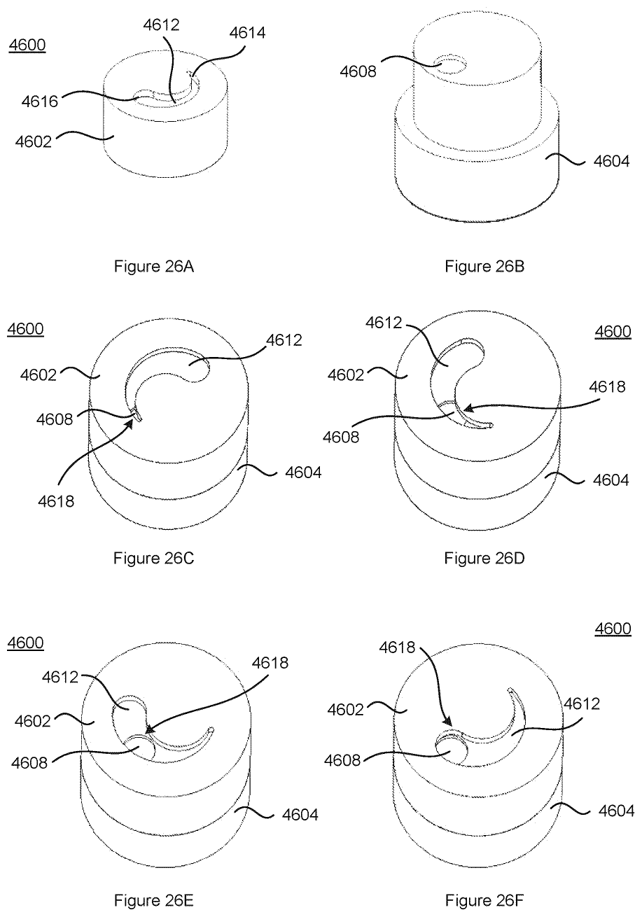

As illustrated in FIGS. 26A to 26F, the flow restriction adjustment mechanism 4600 according to yet another embodiment includes a terminal portion 4602 removably and/or moveably attached to an adapter end portion 4604. The adapter end portion 4604 may be integral with, or separately provided for attachment to, the hollow body (e.g. 4004) of an adapter in accordance with any one of the adapters described herein. The adapter end portion 4604 may be upstream of, or may comprise, the access passage(s).

The terminal portion 4602 is in the form of a cap configured for moveable engagement relative to the adapter end portion 4604. It will be appreciated that any suitable engagement mechanism may be used. For example, press fit engagement, clipping engagement and the like. Typically, the cap 4602 is mounted on the adapter end portion 4604 such that the cap 4602 is rotatable relative to the adapter end portion 4604. A lateral distance between the cap 4602 and the adapter end portion 4604 may be fixed.

The cap 4602 defines an elongate arcuate opening 4612 having a narrow end 4614 and a broad end 4616 in which a width of the opening continuously increases from the narrow end 4614 to the broad end 4616. The adapter end portion 4604 provides an aperture 4608. As more clearly shown in FIGS. 26C to 26F, rotation of the cap 4602 relative to the adapter end portion 4604 aligns the aperture 4608 with different portions along the elongate opening 4612 such that an effective size of an inlet 4618 of an adapter associated with the flow restriction adjustment mechanism 4600 is defined by the combination of the aperture 4612 and a corresponding aligned portion of the elongate opening 4612. In particular, a smaller inlet 4618 is provided when the aperture 4608 is aligned with or proximate the narrow end 4614 of the elongate opening 4612. Similarly, a larger inlet 4618 is provided when the aperture 4618 is aligned with or proximate the broad end 4616 of the elongate opening 4612. Alignment of the aperture 4618 with any suitable section of the elongate opening 4612 between the narrow and broad ends 4614, 4616 provides a variable sized inlet 4618 for the flow restriction adjustment mechanism 4600.

Typically, the aperture 4608 is larger or corresponds in size to the broad end 4616 of the elongate opening 4612 so that the variations in the effective size of the inlet 4618 can be defined by the interaction and relative positions between the aperture 4608 and the elongate opening 4612. In some embodiments, the aperture 4608 can be provided on the cap 4602 instead of the adapter end portion 4604, and the arcuate elongate opening 4612 can be provided on the adapter end portion 4604 instead of the cap 4602. The flow restriction adjustment mechanism would function in a similar manner to that described herein with reference to FIGS. 26A to 26F.

The flow restriction adjustment mechanism 4600 may provide continuous adjustment in the effective size of the inlet 4618 or discrete adjustment in the effective size of the inlet 4618. To provide discrete adjustment, alignment markers and/or locking mechanisms may be provided on the cap 4602 and adapter end portion 4604, for example as shown in FIGS. 30A to 30D as described below.

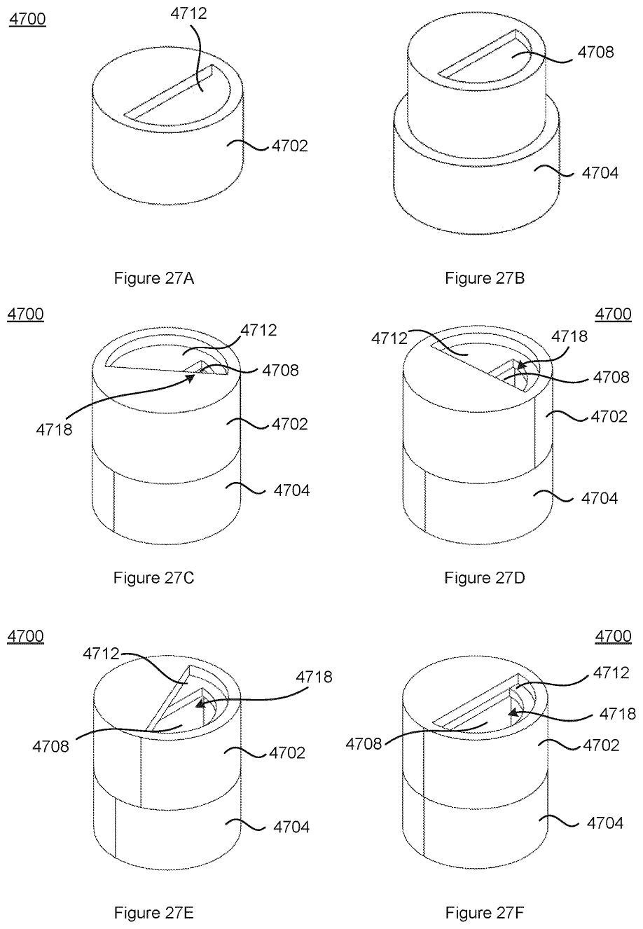

As illustrated in FIGS. 27A to 27F, the flow restriction adjustment mechanism 4700 according to yet another embodiment includes a terminal portion 4702 removably and/or moveably attached to an adapter end portion 4704. The adapter end portion 4704 may be integral with, or separately provided for attachment to, the hollow body (e.g. 4004) of an adapter in accordance with any one of the adapters described herein. The adapter end portion 4704 may be upstream of, or may comprise, the access passage(s).

The terminal portion 4702 is in the form of a cap configured for moveable engagement relative to the adapter end portion 4704. It will be appreciated that any suitable engagement mechanism may be used. For example, press fit engagement, clipping engagement and the like. Typically, the cap 4702 is mounted on the adapter end portion 4704 such that the cap 4702 is rotatable relative to the adapter end portion 4704. A lateral distance between the cap 4702 and the adapter end portion 4704 may be fixed.

The cap 4702 defines a first opening 4712, and the adapter end portion 4704 defines a second opening 4708. As more clearly shown in FIGS. 27C to 27F, rotation of the cap 4702 relative to the adapter end portion 4704 changes the alignment between the first and second openings 4712, 4708 such that an effective size of an inlet 4718 of an adapter associated with the flow restriction adjustment mechanism 4700 is defined by an overlapping opening portion between the first and second openings 2712, 4708.

In particular, as shown in FIG. 27F, a maximum sized inlet 4718 is provided when the first opening 4712 and the second opening 4708 are generally aligned so that the effective size of the inlet 4718 in the position shown in FIG. 27F is defined by the size of the openings 4712, 4718 if the openings are of the same size, or the smaller one of the first and second openings 4712, 4708. As the cap 4702 is rotated relative to the adapter end portion 4704, the two openings 4718, 4712 can be moved in various degrees of mis-alignment such that the effective size of the inlet 4718 defined by an area that the openings 4712, 4708 overlap is reduced from the maximum shown in FIG. 27F. In FIG. 27C, a smaller inlet 4718 can be provided.

The flow restriction adjustment mechanism 4700 may provide continuous adjustment in the effective size of the inlet 4718 or discrete adjustment in the effective size of the inlet 4718. To provide discrete adjustment, alignment markers may be provided on the cap 4702 and adapter end portion 4704, for example as shown in FIGS. 30A to 30D as described below.

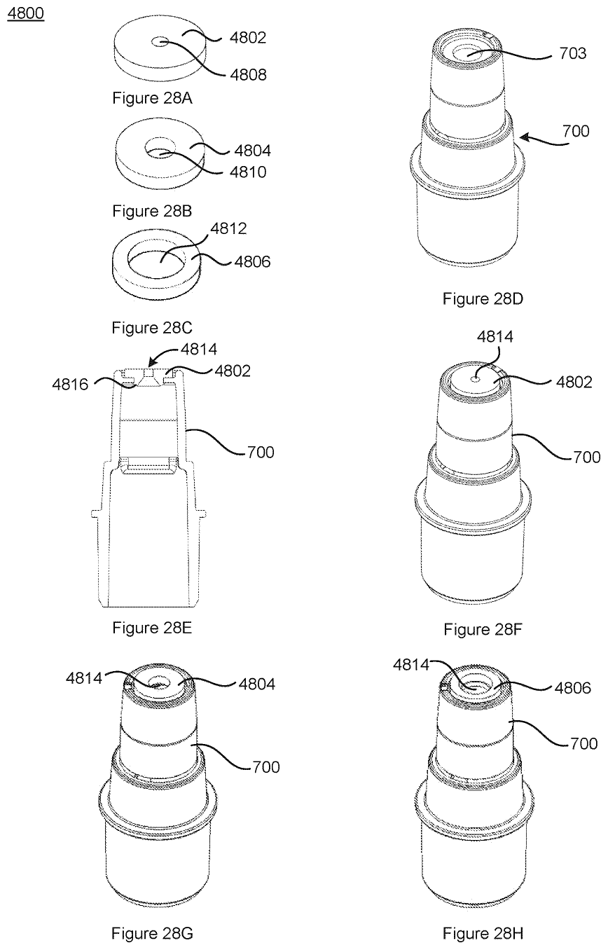

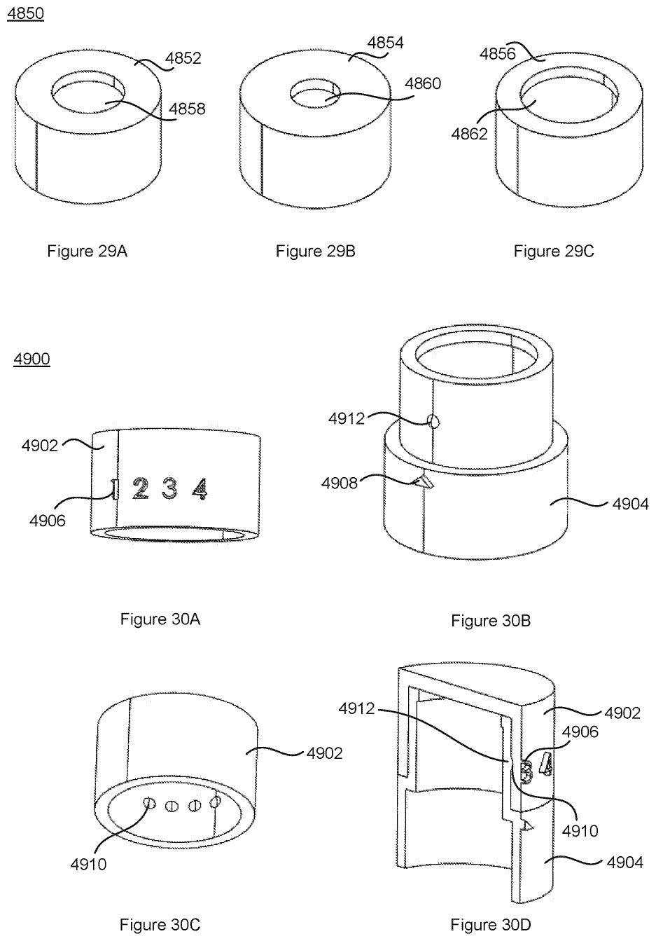

As illustrated in FIGS. 28A to 28H, the flow restriction adjustment mechanism 4800 according to a further embodiment includes one or more terminal portions 4802, 4804, 4806, each terminal portion 4802, 4804, 4806 defining a respective opening 4808, 4810, 4812 therein. The openings 4808, 4810, 4812 vary in size so as to alter the size of an effective inlet 4814 of an adapter 700 associated with the flow restriction adjustment mechanism 4800 as more clearly shown in FIGS. 28E to 28H.

In the embodiment shown, the terminal portions 4802, 4804, 4806 may each include an insertable base 4816 for insertion into the inlet opening 703 of the adapter 700. As more clearly shown in FIGS. 28E to 28H, a respective terminal portion 4802, 4804, 4806 can be secured to an inlet end of the adapter 700 when the corresponding insertable base 4816 is removably received in the inlet opening 703 of the adapter 700. The attached terminal portion 4802, 4804, 4806 may be interchangeable to provide the adjustable flow restriction.

Typically, a terminal portion 4802, 4804, 4806 provides a smaller opening 4808, 4810, 4812 than the inlet opening 703 of the associated adapter 700. Accordingly, mounting a terminal portion 4802, 4804, 4806 to the adapter 700 allows a clinician to reduce the effective inlet 4814 of the adapter 700. Any number of terminal portions 4802, 4804, 4806 each with varying sized openings 4808, 4810, 4812 can be provided to allow a desired flow restriction to be achieved.

A further embodiment of a flow restriction adjustment mechanism 4850 is illustrated in FIGS. 29A to 29C. The flow restriction adjustment mechanism 4850 includes one or more terminal portions 4852, 4854, 4856, each terminal portion 4852, 4854, 4856 defining a respective opening 4858, 4860, 4862 therein. The openings 4858, 4860, 4862 vary in size so as to alter the size of an effective inlet of an adapter (e.g. 700) associated with the flow restriction adjustment mechanism 4850. The concept of the flow restriction adjustment mechanism 4850 operates in a similar manner to the flow restriction adjustment mechanism 4800 described above with reference to FIGS. 28A to 28H. However, each terminal portion 4852, 4854, 4856 is shaped like a cap for mounting over the inlet end of an associated adapter 700. Any suitable mounting mechanism may be used, for example, a respective terminal portion 4852, 4854, 4856 may be mounted to the adapter 700 via threaded engagement, press fit and the like, or a combination thereof.

As previously mentioned, it may be desirable for the flow restriction adjustment mechanism (e.g. 4100, 4200, 4300, 4600, 4700) to provide discrete adjustment of the effective inlet size and/or configuration for an associated adapter. This discrete adjustment may be achieved in a number of ways, for example using different alignment and/locking mechanisms to provide an indication of a desired relative position between the terminal portion/cap (e.g. 4102, 4202, 4302, 4602, 4702) and the respective adapter end portion (e.g. 4102, 4202, 4302, 4602, 4702). FIGS. 30A to 30D illustrate one such example alignment and locking mechanism 4900.

The alignment and locking mechanism 4900 includes markings, for example in the form of numerals 4906 on the terminal portion/cap 4902, and a position indicator 4908 on the adapter end portion 4904. Alignment between each numeral 4906 and the position indicator 4908 may provide a discrete relative position between the cap 4902 and the adapter end portion 4902 to provide an effective inlet of a predetermined size and/or configuration to provide the desired performance characteristics for an associated adapter 700.

In addition, a plurality of recesses 4910 may be provided on an internal surface of the cap 4902 for receiving a protrusion 4912 on the adapter end portion 4904. Each recess 4910 corresponds to and is aligned with a numeral 4906 on the external face of the cap 4902, and the protrusion 4912 is aligned with the position indicator 4908 on the adapter end portion 4904. As the cap 4902 is rotated relative to the adapter end portion 4904, the protrusion 4912 is received by a respective recess 4910 when a corresponding numeral 4906 is aligned with the position indicator 4908 so as to facilitate correct positioning of the cap 4902 relative to the adapter end portion 4904. As more clearly illustrated in the cross-sectional view in FIG. 30D, the numeral ‘3’ is aligned with the position indicator 4908 and a corresponding recess 4910 receives the protrusion 4912 therein. In use, a force can be applied to the cap 4902 to move the protrusion 4912 to an adjacent recess 4910 to change the adjustment setting, for example from 3 to 4 as indicated by the marking 4906.

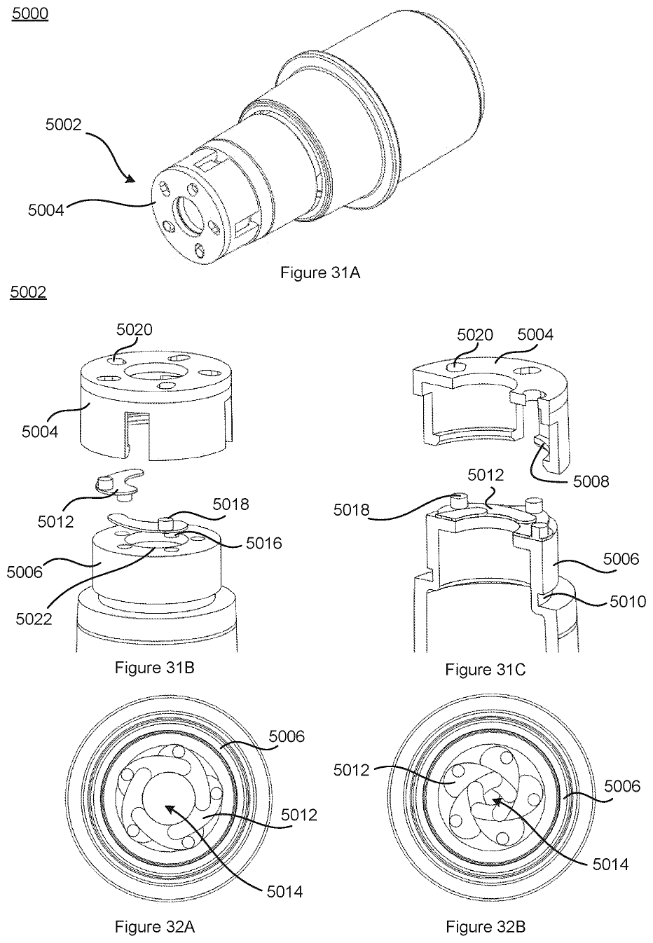

An adapter 5000 having a flow restriction adjustment mechanism 5002 according to a further embodiment is illustrated in FIGS. 31A to 34C.

As shown in FIG. 31A, the flow restriction adjustment mechanism 5002 is provided proximate an inlet end of the of the adapter 5000. The flow restriction adjustment mechanism 5002 includes a terminal portion 5004 moveably coupled to an adapter end portion 5006 of the adapter 5000. In particular, the terminal portion 5004 includes a lip 5008 which is receivable within a groove 5010 of the adapter end portion 5006 so as to couple the terminal portion 5004 to the adapter end portion 5006 whilst allowing rotational movement of the terminal portion 5004 relative to the adapter end portion 5006.

The flow restriction adjustment mechanism 5002 further includes a plurality of blades 5012. The blades 5012 cooperate with one another to widen (FIG. 32A) or narrow (FIG. 32B) the size of an effective inlet 5014 for the adapter 5000. Whilst five blades 5012 are illustrated in the particular embodiment shown in FIGS. 32A, 32B, and FIGS. 34A to 34C, it is to be understood any suitable number of blades can be provided without departing from the scope of the embodiment.

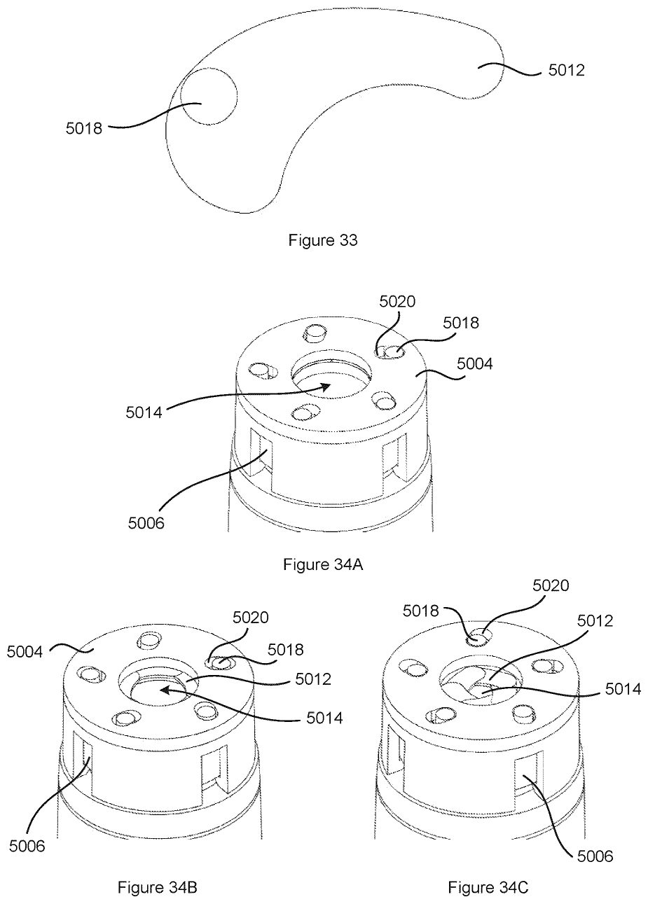

Each blade 5012 is generally arcuate shaped (as more clearly shown in FIG. 33) and is pivotally mounted to the adapter end portion 5006 via pivot attachment point 5016 as shown in FIG. 31B. As more clearly illustrated in FIGS. 32A and 32B, each of the blades 5012 is pivotable about its respective pivot attachment point 5016 so as to maintain the inlet opening 5022 of the adapter unobstructed (e.g. see FIGS. 32A, 34A), or partially obstructed (e.g. see FIGS. 32B, 34B and 34C).

Each blade 5012 further includes a protrusion 5018 which can be slidable received within a corresponding slot 5020 in the terminal portion 5004. The slot 5020 serves to limit the range of pivotal movement for each blade 5012. As more clearly illustrated in FIGS. 34A to 34C, rotation of the terminal portion 5004 relative to the adapter end portion 5006 causes the protrusion 5018 of each blade 5012 to slide between opposite ends of the corresponding slot 5020.

When the protrusion 5018 is positioned against an outer end of the slot 5020 as shown in FIG. 34A, each of the blades 5012 are pivoted away from a centre of the of the inlet opening 5022 providing little or no obstruction of the inlet opening 5022, thereby providing a maximum (largest) effective inlet 5014 for the adapter 5000. When the protrusion 5018 is positioned against an inner end of the slot 5020 (opposite the outer end of the slot 5020) as shown in FIG. 34C, each of the blades 5012 is pivoted towards a centre of the of the inlet opening 5022 providing maximum obstruction of the inlet opening 5022, thereby providing a minimum (smallest) effective inlet 5014 for the adapter 5000. When the protrusion 5018 is positioned at an intermediate position between opposite ends of the slot 5020 as shown in FIG. 34B, each of the blades 5012 are pivoted to a position where each of the blades 5012 partially obstructs the inlet opening 5022 such that an intermediate (medium sized) effective inlet 5014 is provided for the adapter 5000.

The flow restriction adjustment mechanism 5000 can provide continuous adjustment of the size of the effective inlet 5014 by moving the protrusion 5018 to any suitable position within the slot 5020. Alternatively, discrete adjustment of the size of the effective inlet 5014 may be provided by using alignment and/or locking mechanisms similar to the example previously described with reference to FIGS. 30A to 30D.

### Interpretation

This specification, including the claims, is intended to be interpreted as follows:

The terms ‘conduit’ and ‘tubing’ as used in this specification and claims are intended to broadly mean, unless the context suggests otherwise, any member that forms or provides a lumen for directing a flow of liquid or gases. For example, a conduit or conduit portion may be part of a humidification device, or may be a separate conduit attachable to a humidification device to provide a flow of fluid or a fluid communication.

In various texts, such as those incorporated herein by reference, embodiments of the ‘adapter’ may be referred to generically as a ‘connector’. Accordingly, depending on the context, the word ‘connector’ may be taken to refer to an ‘adapter’, and vice versa.

The terms ‘comprising’ and/or ‘including’ as used in this specification and claims means ‘consisting at least in part of’. When interpreting each statement in this specification and claims that includes the term ‘comprising’ and/or ‘including’, features other than that or those prefaced by the term may also be present. Related terms such as ‘comprise’ and ‘comprises’, ‘include’ and ‘includes’ are to be interpreted in the same manner.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

As used herein ‘(s)’ following a noun means the plural and/or singular forms of the noun.

The terms “a” and “an” mean “one or more”, unless expressly specified otherwise.

Neither the title nor the abstract of the present application is to be taken as limiting in any way as the scope of the claimed invention.

Where the preamble of a claim recites a purpose, benefit or possible use of the claimed invention, it does not limit the claimed invention to having only that purpose, benefit or possible use.

It should be noted that terms of degree such as “generally”, “substantially”, “about” and “approximately” as used herein mean a reasonable amount of deviation of the modified term such that the end result is not significantly changed. These terms of degree should be construed as including a deviation of the modified term if this deviation would not negate the meaning of the term it modifies.

Embodiments or examples described in the specification are intended to be illustrative of the invention, without limiting the scope thereof. The invention is capable of being practised with various modifications and additions as will readily occur to those skilled in the art. Accordingly, it is to be understood that the scope of the invention is not to be limited to the exact construction and operation described or illustrated, but only by the following claims.

The mere disclosure of a method step or product element in the specification should not be construed as being essential to the invention claimed herein, except where it is either expressly stated to be so or expressly recited in a claim.

The disclosure of any document referred to herein is incorporated by reference into this patent application as part of the present disclosure, but only for purposes of written description and enablement and should in no way be used to limit, define, or otherwise construe any term of the present application where the present application, without such incorporation by reference, would not have failed to provide an ascertainable meaning. Any incorporation by reference does not, in and of itself, constitute any endorsement or ratification of any statement, opinion or argument contained in any incorporated document.

The terms in the claims have the broadest scope of meaning they would have been given by a person of ordinary skill in the art as of the relevant date.

1. An adapter comprising
a rigid base portion defining an outlet, and
a head portion defining an inlet, the head portion being configured for removable attachment to the rigid base portion such that a gas flow passage is provided between the inlet and the outlet when the head portion is attached to the rigid base portion,
wherein the adapter is configured to provide
a first flow restriction for restricting gas flow through the gas flow passage, and
an access passage to the gas flow passage, the access passage being provided downstream of the first flow restriction.

2. The adapter of claim 1, wherein the access passage is provided by an aperture in the head portion.

3. The adapter according to any one of the preceding claims, wherein the first flow restriction is provided at the inlet.

4. The adapter according to any one of the preceding claims, wherein the head portion is tethered to the rigid base portion.

5. The adapter according to any one of the preceding claims, wherein the head portion is tapered.

6. The adapter according to any one of the preceding claims, wherein the head portion is configured for sealing engagement with a connector associated with a pressure valve.

7. The adapter according to claim 6, wherein the head portion is configured to form a cavity with the connector when the head portion is engaged with the connector.

8. The adapter of claim 7, wherein the cavity is in fluid communication with the gas flow passage via the access passage.

9. The adapter according to any one of the preceding claims, wherein the rigid base portion includes one or more projections for engagement with one or more corresponding recesses provided by the head portion, for removable attachment thereto.

10. The adapter according to any one of the preceding claims, further comprising
a second head portion configured for removable attachment to the rigid base portion, the second head portion defining an inlet such that a gas flow passage is provided between the inlet of the second head portion and the outlet of the rigid base portion when the second head portion is attached to the rigid base portion,
wherein the adapter is configured to provide a second flow restriction for restricting gas flow through the gas flow passage when the second head portion is attached to the base portion, and
wherein the second flow restriction is different to the first flow restriction.

11. The adapter of claim 10, wherein the adapter is configured to provide an access passage to the gas flow passage when the second head portion is attached to the rigid base portion, the access passage being provided downstream of the second flow restriction.

12. The adapter of claim 10 or 11, wherein the inlet of the second head portion is smaller than the inlet of the first head portion.

13. An adapter comprising
a hollow body defining an inlet and an outlet configured to provide a gas flow passage therethrough,
a flow restriction configured to restrict gas flow through the gas flow passage,
an access passage to the gas flow passage, the access passage being provided downstream of the flow restriction, and
an attachment portion proximate the outlet configured for removable attachment to a rigid adapter base portion.

14. The adapter of claim 13, wherein the hollow body defines an aperture downstream of the inlet for providing the access passage to the gas flow passage.

15. The adapter of claim 13 or 14, wherein the flow restriction is provided at the inlet.

16. The adapter of any one of claims 13 to 15, wherein the hollow body is tapered towards the inlet.

17. The adapter of claim 14, further including an engagement portion configured for sealing engagement with a connector associated with a pressure valve.

18. The adapter of claim 17, wherein the hollow body is configured to form a cavity with the connector when the engagement portion is engaged with the connector.

19. The adapter of claim 18, wherein the cavity is in fluid communication with the gas flow passage via the access passage.

20. The adapter of any one of claims 13 to 19, wherein the attachment portion includes one or more recesses for engagement with one or more projections of the base portion for removable attachment thereto.

21. An adapter assembly including
one or more adapters, each adapter being an adapter according to any one of claims 13 to 20,
a rigid adapter base portion for removable attachment to the one or more adapters,
wherein the flow restriction of each adapter is different.

22. The adapter assembly of claim 21, the inlet size of each adapter is different.

23. The adapter assembly of claim 21 or 22, further including a tether for tethering one or more of the adapters to the base portion.

24. The adapter assembly of claim 23, wherein each of the one or more adapters and the rigid base portion includes a channel for receiving the tether.

25. An adapter comprising
a hollow body defining an inlet and an outlet for providing a gas flow passage therethrough,
a flow restriction for restricting gas flow through the gas flow passage,
an access passage to the gas flow passage, the access passage being provided downstream of the flow restriction, and
a mounting portion proximate the outlet for removable mounting to a second like adapter.

26. The adapter of claim 25, wherein the hollow body defines an aperture downstream of the inlet for providing the access passage to the gas flow passage.

27. The adapter of claim 25 or 26, wherein the hollow body includes a stepped portion such that a first internal cross-sectional area of the hollow body immediately upstream of the stepped portion is smaller than a second internal cross-sectional area of the hollow body immediately downstream of the stepped portion, and wherein the aperture is provided in the stepped portion.

28. The adapter of claim 27, wherein the mounting portion has a third internal cross-sectional area which is greater than the first and second internal cross-sectional areas for receiving the second like adapter.

29. The adapter of claim 28, wherein the mounting portion is configured for sealing engagement with a corresponding hollow body of the second like adapter proximate a corresponding stepped portion of the second like adapter.

30. The adapter of any one of claims 25 to 29, the mounting portion is configured such that a corresponding inlet and access passage aperture of the second like adapter are fully encapsulated in the hollow body of the adapter when the adapter is mounted on the second like adapter.

31. The adapter of any one of claims 25 to 30, wherein the flow restriction is different to the flow restriction provided by the second like adapter.

32. The adapter of claim 31, wherein the flow restriction is more restrictive than the flow restriction provided by the second like adapter.

33. The adapter of claim 32, and the inlet of the adapter is smaller than the inlet of the second like adapter to provide the more restrictive flow restriction.

34. The adapter of any one of claims 25 to 33, wherein the mounting portion includes a flared portion proximate the outlet of the hollow body for receiving the second like adapter.

35. The adapter of any one of claims 25 to 33, wherein the mounting portion includes a stepped flange proximate the outlet of the hollow body for receiving the second like adapter.

36. The adapter of any one of claims 25 to 35, further including an engagement portion configured for sealing engagement with a connector associated with a pressure valve.

37. The adapter of claim 36, wherein the hollow body is configured to form a cavity with the connector when the engagement portion is engaged with the connector.

38. The adapter of claim 37, wherein the cavity is in fluid communication with the gas flow passage via the access passage.

39. An adapter comprising
a hollow body defining an inlet and an outlet configured to provide a gas flow passage therethrough,
a flow restriction configured to restrict gas flow through the gas flow passage,
wherein the flow restriction is adjustable.

40. The adapter of claim 39, wherein the adapter further includes an access passage to the gas flow passage, the access passage being provided downstream of the flow restriction.

41. The adapter of claim 39 or 40, further including a flow restriction adjustment mechanism for adjusting the flow restriction.

42. The adapter of claim 41, wherein the flow restriction adjustment mechanism includes a terminal portion movably mounted to an inlet end of the adapter such that relative movement of the terminal portion relative to the inlet end of the adapter adjusts a size of the flow restriction.

43. The adapter of claim 41, wherein the flow restriction adjustment mechanism includes an insert receivable by the inlet of the adapter for reducing a size of the flow restriction.
